(19) United States
(12) Patent Application Publication
(10) Pub. No.: US 2022/0323425 A1
(43) Pub. Date: Oct. 13, 2022

(54) CO-POTENTIATORS FOR THERAPY OF CYSTIC FIBROSIS CAUSED BY MINIMAL FUNCTION CFTR MUTANTS

(71) Applicant: THE REGENTS OF THE UNIVERSITY OF CALIFORNIA, Oakland, CA (US)

(72) Inventors: Alan S. VERKMAN, Oakland, CA (US); Puay-Wah PHUAN, Oakland, CA (US); Peter M. HAGGIE, Oakland, CA (US)

(21) Appl. No.: 17/639,311

(22) PCT Filed: Aug. 28, 2020
(86) PCT No.: PCT/US2020/048645

§ 371 Date: Feb. 28, 2022

Related U.S. Application Data
(60) Provisional application No. 62/893,107, filed on Aug. 28, 2019.

(51) Int. Cl. A61K 31/438; A61K 31/47; A61K 45/06; A61K 31/4245

(52) U.S. Cl. CPC A61K 31/438; A61K 31/47; A61K 45/06; A61K 31/4245

(57) ABSTRACT

Provided herein are combination-potentiator (“co-potentiator”) therapeutic regimens, which can be used to modulate cystic fibrosis transmembrane conductance regulator (CTFR) mutant proteins. Co-potentiators have potential utility for treatment of many loss-of-function mutations of the CFTR chloride channel (e.g., N1303K).

### STATEMENT OF GOVERNMENT INTEREST

This invention was made with government support under Grant No. P30 DK072517 awarded by the National Institutes of Health. The government has certain rights in the invention.

### BACKGROUND

### Technical Field

This disclosure is related to potent potentiators for use in treating cystic fibrosis caused by minimal function fibrosis transmembrane conductance regulator (CFTR) mutants.

### Description of the Related Art

Cystic fibrosis (CF) is caused by loss of function mutations in the CFTR gene that affect the production of the CFTR protein, a cAMP-activated chloride channel. More than 2000 CF-causing CFTR gene mutations have been identified (1).

Small-molecule CFTR modulators have been developed that rescue defective cellular processing and cell-surface targeting of mutant CFTRs (correctors) or rescue defective channel gating to restore CFTR anion transport (potentiators). (1-3) The potentiator Kalydeco (ivacaftor/VX-770) has been approved for CF subjects with gating mutations, including G551D-CFTR and now 38 additional mutations (2). The corrector/potentiator combinations Orkambi (VX-770 plus lumacaftor/VX-809) and Symdeko (VX-770 plus tezacaftor/VX-661) have been approved for CF subjects that are homozygous for the most common CF-causing CFTR mutation, F508del, or who have one F508del allele and a residual function CFTR mutation (2). Triple drug combinations, consisting of two correctors and one potentiator, have shown additional benefits in clinical trials and may soon be approved for CF subjects with one F508del allele and a second CFTR allele carrying any mutation (2, 4-6). Current and improved so called “next-generation” therapeutics are promising for treating up to 90% of all CF subjects (2).

However, certain CFTR mutations (about 10% of CF subjects) appear to be refractory to available potentiators and correctors. Non-responsive minimal function CFTR mutations are distributed throughout the CFTR protein and are associated with low CFTR function due to defective channel processing, cell-surface trafficking, and/or channel gating. One such minimal function CFTR mutation is N1303K, a missense point mutation located in nucleotide binding domain 2 (NBD2), which is the 5^{th} most common CFTR mutation worldwide accounting for ˜2.5% of CFTR mutations (www.cftr2.org). Other minimal function missense CFTR mutants are found in membrane spanning domain (MSD) 1, including G85E and R334W, and MSD2, including L1077P and M1101K. These four CFTR mutants are found in ˜1.4% of ˜88,000 CF subjects in the CFTR2 database. Premature termination codon (PTC) mutations also have no available therapy, including G542X located in NBD1 and W1282X located in NBD2, which are the 2^{nd} and 4^{th} most common CFTR mutations (5% and 4% allele frequency in CFTR2 database, respectively).

It has been previously reported that VX-770, when used in combination with a second potentiator (ASP-11), increased chloride channel function of N1303K-CFTR and the truncated W1282X-CFTR protein product by ˜8-fold compared with VX-770 alone (9, 10). This combination potentiator (or co-potentiator) approach was also shown to be effective in increasing the chloride channel function of G551D-CFTR, with ˜50% improvement compared with VX-770 alone (10).

There remains a need in the art for improved therapy for treating cystic fibrosis caused by minimal function CFTR mutations.

### BRIEF SUMMARY

Provided herein are methods of using co-potentiators for treating CF subjects having minimal function CFTR mutants and methods of identifying by high-throughput screening, novel co-potentiator scaffolds with nanomolar potency.

As discussed in more detail herein, the co-potentiators are classified into two classes depending on their respective mechanisms of action on the CFTR protein. Briefly speaking, Class I potentiators include the classical potentiators such as VX-770 or GLPG1837 (see FIG. 3A), which rescue the defective gating of CFTR mutations in NBD2. Class II potentiators are identified by the high-throughput screening disclosed herein, and are believed to bind to CFTR in a manner that stabilizes partial or misfolded NBD2 structurally or thermodynamically. Because potentiators may produce both long-range and local conformational changes, as well as thermodynamic alterations to rescue defective conductance of mutant CFTRs, potentiator synergy screening as disclosed herein may be useful for selected mutations in other regions of the CFTR protein.

One embodiment thus provides a method of treating cystic fibrosis in a CF subject having at least one missense, nonsense, deletion, or truncation mutation, the method comprising administering at least one Class I potentiator and at least one Class II potentiator to the subject, wherein the Class II potentiator has one of the following structures (A), (B), (C), or (D):

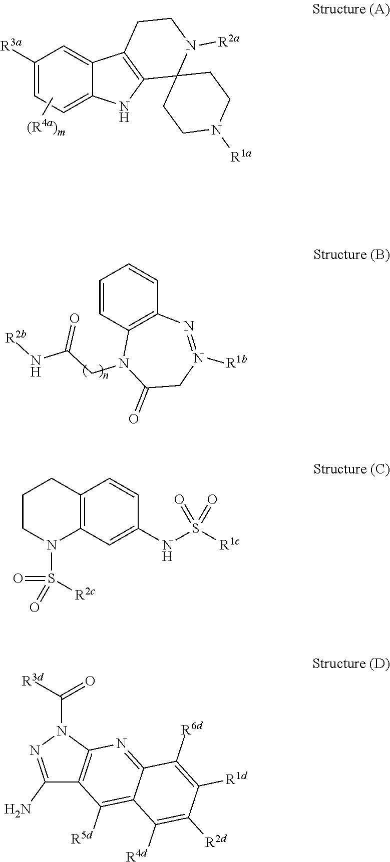

wherein:

m, n, R^{1a}, R^{2a}, R^{3a}, R^{4a}, R^{1b}, R^{2b}, R^{1c}, R^{2c}, R^{1d}, R^{2d}, R^{3d}, R^{4d}, R^{5d}, and R^{6d} are as defined herein.

### BRIEF DESCRIPTION OF THE DRAWINGS

In the figures, identical reference numbers identify similar elements or acts. The sizes and relative positions of elements in the figures are not necessarily drawn to scale. For example, the shapes of various elements and angles are not drawn to scale and some of these elements are enlarged and positioned to improve figure legibility. Further, the particular shapes of the elements as drawn, are not intended to convey any information regarding the actual shape of the particular elements, and have been solely selected for ease of recognition in the figures.

FIGS. 1A-1D illustrate known co-potentiator ASP-11 activity in minimal function CFTR mutants in transfected FRT cells. FIG. 1A. Structure of ASP-11. FIG. 1B. Location of minimal function CFTR mutants studied herein. FIG. 1C. Short-circuit current in FRT cells expressing I1234del-CFTR, Q1313X-CFTR and S492F-CFTR in response to 20 μM forskolin (fsk), 5 μM VX-770, 20 μM ASP-11 and 10 μM CFTRᵢₙₕ-172. As indicated, cells were treated with 3 μM VX-809 for 18-24 h prior to measurements. FIG. 1D. Short-circuit current in FRT cells expressing predicted W1282X-CFTR read-through products W1282L-CFTR, W1282R-CFTR and W1282C-CFTR (without and with VX-809 pretreatment). Data representative of 3 replicates.

FIG. 2 shows a summary of short-circuit current responses to modulators for CFTR mutants in transfected FRT cells. Responses to indicated CFTR mutants with 20 μM forskolin, 5 μM VX-770, 20 μM ASP-11 (with or without 3 μM VX-809 overnight). For reference, data shown for cells expressing wild type CFTR. Mean±S.E.M., n=3.

FIGS. 3A-3D illustrate the definitions for Class I and Class II CFTR potentiators. FIG. 3A. Chemical structures of Class I and II potentiators. FIG. 3B. Short-circuit current in FRT cells expressing N1303K-CFTR in response to forskolin and indicated potentiators. Concentrations: 20 μM forskolin, 5 μM VX-770, 20 μM ASP-11, 20 μM GLPG1837, 20 μM P2, 20 μM P3, 20 μM P5, 20 μM W1282Xₚₒₜ-C01, and 10 μM CFTRᵢₙₕ-172. FIG. 3C. Short-circuit current in FRT cells expressing Q1313X-CFTR in response to forskolin and indicated potentiators. Concentrations: as in panel B, and 25 μM apigenin. Data representative of 3 replicates. FIG. 3D. Summary of relative N1303K-CFTR activation in response to combinations of Class I and Class II potentiators. Mean±S.E.M., n=3.

FIGS. 4A-4E show co-potentiators identified by high-throughput screening. FIG. 4A. Assay design. FIG. 4B. Summary of screening workflow and results. FIG. 4C. Chemical structures of novel co-potentiators identified by screening.

FIG. 4D. Short-circuit current in FRT cells expressing W1282X-CFTR. Concentrations: 20 μM forskolin (fsk), 5 μM VX-770. FIG. 4E. Effects of sequential addition of 20 μM forskolin (fsk) followed by 20 μM of A-01, B-01, C-01 and D-01, and 5 μM of VX-770 in FRT cells expressing W1282X-CFTR. CFTRᵢₙₕ-172 was used at 10 μM in all experiments. Data representative of 3 replicates.

FIGS. 5A-5D show structure-activity analysis of pyrazoloquinoline and spiro[piperidine-4,1pyrido[3,4-b]indoles] co-potentiators. FIG. 5A. Structural determinants of pyrazoloquinoline (CP-Axxx) and FIG. 5B. spiro[piperidine-4,1pyrido[3,4-b]indoles] (CP-Dxxx) co-potentiator activity. FIG. 5C. Short-circuit current in FRT cells expressing N1303K-CFTR showing responses to 20 μM forskolin (fsk), 5 μM VX-770, and indicated concentrations of CP-A061 (left) and CP-D123 (center). (right) Summary of concentration-dependence data. FIG. 5D. Short-circuit current in FRT cells expressing Q1313X-CFTR done as in panel C. Curves representative of three replicates, summary data shown as mean±S.E.M., n=3.

FIGS. 6A-6B show N1303K-CFTR activity in human airway epithelial cell cultures. FIG. 6A. Short-circuit current in gene-edited 16HBE14o-cells expressing N1303K-CFTR. Concentrations: 20 μM amiloride, 20 μM forskolin, 5 μM VX-770, 20 μM CP-A061 and CP-D123, and 10 μM CFTRᵢₙₕ-172. Representative original curves (left and center) and summary of changes in current (ΔIsc) (right). Mean±S.E.M., n=3, *P<0.01). FIG. 6B. Short-circuit current in primary cultures of human bronchial epithelial cells from a homozygous N1303K-CFTR CF subject. Concentrations were as in panel A. Representative original curves (left and center) and summary of changes in current (ΔIsc) (right). Mean±S.E.M., n=3, *P<0.01).

FIGS. 7A-7D show short-circuit current measurement of mutant CFTR activation by novel spiro[piperidine-4,1pyrido[3,4-b]indoles]. FIG. 7A. (left) Short-circuit current data in FRT cells expressing N1303K-CFTR in response to 20 μM forskolin (fsk), 5 μM VX-770, indicated concentration of Compounds 2i and 2e, and 10 μM CFTRᵢₙₕ-172. (right) Summary of concentration-dependence data (n=3, mean±S.E.M.). FIG. 7B. Measurements done as in FIG. 7A, but with 20 μM GLPG1837 instead of VX-770. C. Short-circuit current in FRT cells expressing I1234del-CFTR in response to 20 μM forskolin, 5 μM VX-770, indicated concentration of Compound 2i, and 10 μM CFTRᵢₙₕ-172. FIG. 7D. Short-circuit current in FRT cells expressing R347P-CFTR in response to 20 μM forskolin, 5 μM VX-770, 20 μM Compound 2i and 10 M CFTRᵢₙₕ-172. In all studies, cells were corrected with 3 μM VX-661 for 18-24 hours prior to measurement. All traces are representative of three replicates.

FIGS. 8A-8C demonstrate activity of Compound 2i in human airway epithelial cell cultures. FIG. 8A. Short-circuit current in gene-edited 16HBE14o-cells expressing N1303K-CFTR. FIG. 8B. Short-circuit current data in gene-edited 16HBE14o-cells expressing I1234del-CFTR. FIG. 8C. Summary of changes in short-circuit current (ΔIsc; mean±S.E.M., n=3, *P<0.05). Concentrations: 20 μM forskolin, 5 μM VX-770, 10 μM Compound 2i, and 10 μM CFTRᵢₙₕ-172. 16HBE14o-cell models expressing I1234del-CFTR were corrected with 18 μM VX-661 and 3 μM VX-445 for 18-24 hours prior to measurement.

### DETAILED DESCRIPTION

Disclosed herein are embodiments generally directed to a therapeutic strategy for treating CF subjects, in particular, those with CFTR mutations that do not respond significantly to the available CFTR modulators such as Kalydeco, Orkambi and Symdeko. More specifically, novel co-potentiator scaffolds with drug-like properties and EC₅₀ down to 500 nM or even 300 nM have been identified. It has been observed that compounds of these scaffolds act in synergy with VX-770 or other Class I potentiators to increase CFTR chloride current. The potentiator/co-potentiator paradigm disclosed herein is therefore effective for treating CF subjects having a variety of missense, nonsense, and deletion mutations specifically in NBD2 of CFTR.

### Classification of Potentiators to Predict Synergy

According to various embodiments, CF subjects with certain mutations are treated by a combination therapy comprising at least two distinct potentiators. The potentiators are distinct in their mechanisms of actions (e.g., targeting distinct binding sites or regions on CFTR mutants); and the additive or synergistic effects can benefit CF subjects that do not respond significantly to the available drugs. A classification system defining these distinct potentiators is discussed herein.

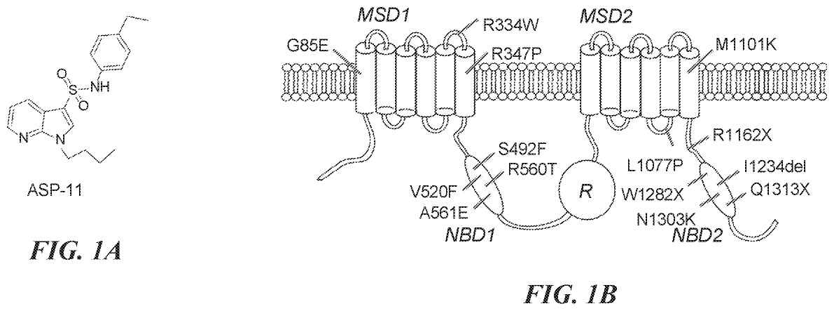

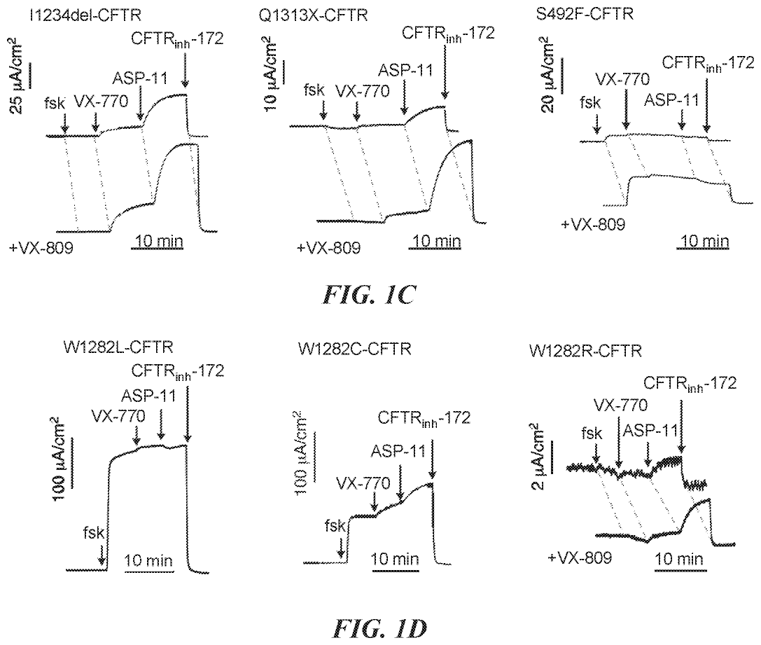

One of the first reported co-potentiators, arylsulfonamide-pyrrolopyridine (ASP-11) (structure shown in FIG. 1A), was found to have increased chloride current by ˜8-fold over that produced by a high concentration of VX-770 in transfected FRT cells expressing N1303K-CFTR or CFTR1281 (the truncation product generated by W1282X-CFTR) (9, 10).

To investigate the CFTR mutational space specificity for ASP-11, 12 additional minimal function CFTR mutants were studied (FIG. 1). The nine missense mutants studied are located throughout the CFTR protein and included mutations in MSD1 (G85E, R334W, R347P), NBD1 (S492F, V520F, R560T, A561E) and MSD2 (L1077P, M1101K). Two C-terminal PTC mutations, R1162X, and Q1313X, were studied based on the prior W1282X-CFTR data showing benefit of co-potentiators (9). Lastly, the complex CFTR mutant c.3700 A>G was also tested, which introduces a point mutation (I1234V-CFTR) that retains CFTR activity, or a cryptic splice site resulting in a 6-amino acid deletion in NBD2, p.Ile1234_Arg1239del (I1234del-CFTR) (11). Testing of these NBD2 truncation and deletion mutations was motivated by biochemical and electrophysiological evidence suggesting that incomplete NBD2, including CFTR polypeptides as short as 1217 amino acids, may sometimes allow partial CFTR cell-surface expression and function (12, 13).

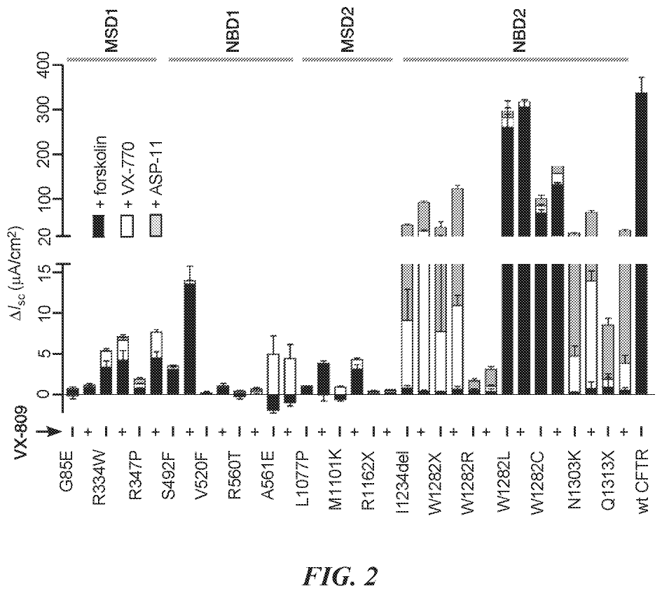

As shown in FIG. 1C and summarized in FIG. 2, ASP-11 acted in synergy with VX-770 to increase chloride current in FRT cells expressing the NBD2 mutations I1234del- and Q1313X-CFTR by ˜3-9-fold compared to VX-770, with 2-3-fold greater currents when cells were pretreated with corrector VX-809. In contrast, the NBD1 mutation S492F-CFTR showed limited forskolin-stimulated current and no response to VX-770 or ASP-11, though VX-809 increased the forskolin response. Qualitatively similar absence of response to ASP-11 was found for other CFTR mutations located in MSD1 (e.g. R334W), NBD1 (V520F and R560T), and MSD2 (L1077P and M1101K) (FIG. 2). In contrast to Q1313X-CFTR, the C-terminus PTC mutant R1162X-CFTR (predicted to truncate CFTR just after MSD2) did not respond to forskolin, VX-770, ASP-11 or VX-809.

Compounds that promote read-through of PTCs to generate full-length CFTR have shown limited efficacy in cell culture studies (14-16). Though the read-through drug ataluren (PTC124) was ineffective in clinical trials (17), newer compounds are in development. The ability of ASP-11 to activate chloride current in predicted W1282X-CFTR read-through products was investigated. G418 action on W1282X was found to insert mainly leucine or cysteine at position 1282 (18), and ataluren to insert arginine (16). FRT cell lines were generated that stably expressing W1282L-, W1282C- and W1282R-CFTR. W1282L-CFTR showed robust forskolin stimulation with little additional VX-770 or ASP-11 effect (FIG. 1D and FIG. 2). W1282C-CFTR showed less forskolin response but significant further effects of VX-770 and ASP-11, and W1282R-CFTR showed minimal function. Together, the data as summarized in FIG. 2 suggest that several missense mutation, deletion and truncation mutations in NBD2 may be amenable to co-potentiator action.

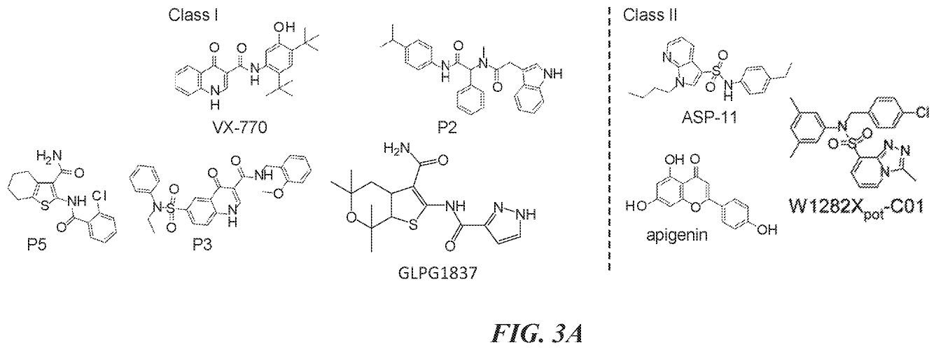

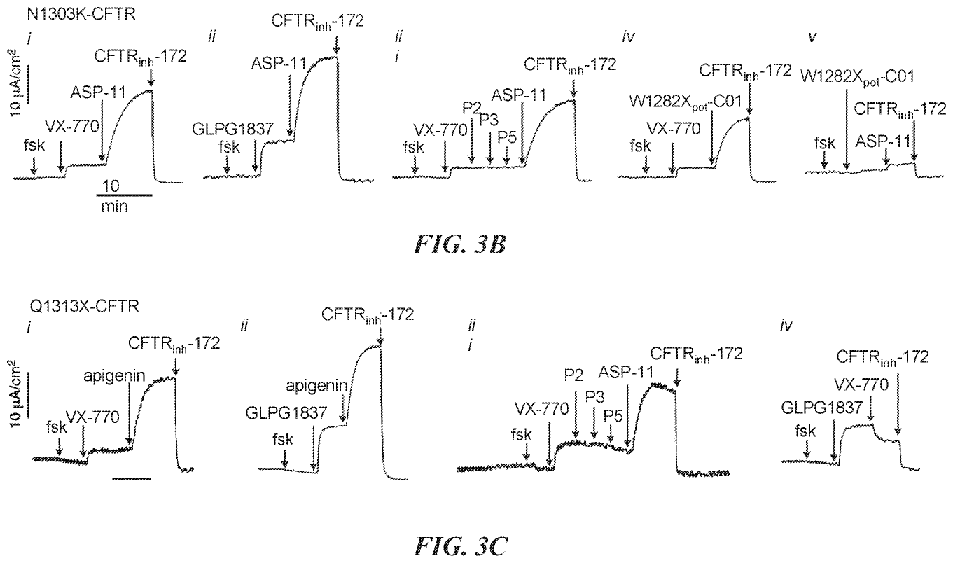

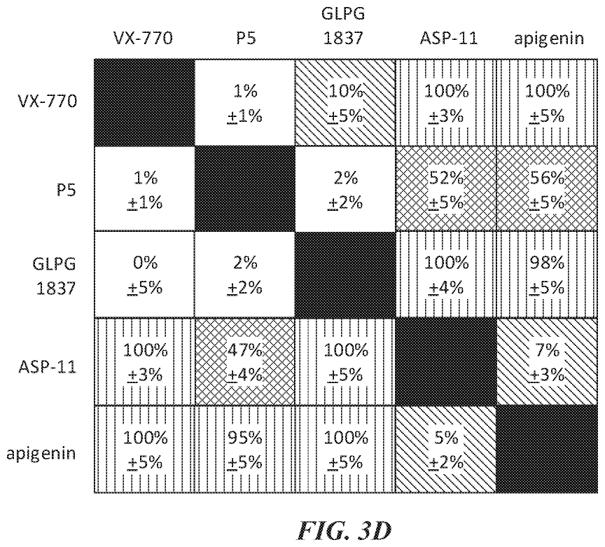

Without wishing to be bound by theory, the applicant hypothesizes that distinct binding sites may be required to bind a potentiator and co-potentiator to rescue channel activity of CFTR mutants. Thus, depending on distinct mechanisms of action or distinct binding sites targeted, CFTR potentiators are classified into Class I and Class II compounds. As an example, potentiator VX-770 is a Class I compound, and co-potentiator ASP-11 is a Class II compound (see also FIG. 3A). Short-circuit current measurements in N1303K-CFTR-expressing FRT cells confirmed synergy when ASP-11 was added after VX-770 (FIG. 3B (i)). Similar results were found for ASP-11 added after GLPG1837 (FIG. 3B (ii)), indicating that GLPG1837 is a Class I potentiator. Additions of P2, P3 and P5 (potentiator activity confirmed in separate studies, not shown) did not further increase current when added after VX-770 (FIG. 3B (iii)), indicating that these compounds also belong to Class I. Interestingly, VX-770 addition after GLPG1837 mildly reduced current (data not shown), which is consistent with competitive binding between VX-770 and GLPG1837 as reported for G551D-CFTR (19). Another co-potentiator previously reported to activate W1282X-CFTR, W1282Xₚₒₜ-C01 (9), activated N1303K-CFTR when used with VX-770 (FIG. 3B (iv)), demonstrating this compound is a Class II potentiator. Finally, two Class II potentiators, W1282Xₚₒₜ-C01 and ASP-11, produced little activation when added together (FIG. 3Bv). In each experiment, CFTRᵢₙₕ-172 confirmed that current was from CFTR.

Studies on FRT cells expressing Q1313X-CFTR produced similar data (FIG. 3C). Addition of the Class I potentiators VX-770 (FIG. 3C (i)) or GLPG1837 (FIG. 3C (ii)) followed by the Class II potentiator apigenin showed synergy. In contrast, sequential additions of Class I potentiators P2, P3 and P5 did not elevate current following VX-770, whereas Class II potentiator ASP-11 did (FIG. 3C (iii)). As seen for N1303K-CFTR and G551D-CFTR (19), VX-770 added after GLPG1837 reduced current in Q1313X-CFTR expressing cells (FIG. 3C (iv)). As summarized in FIG. 3D for N1303K-CFTR, synergy was observed for Class I and Class II compounds used together, but not for combinations of Class I-Class I or Class II-Class II compounds.

### Class I Potentiators

Class I potentiators may include those compounds that thermodynamically alter the closed-open equilibrium of CFTR via binding to the CFTR mutants. VX-770 is a classic example of Class I potentiators. VX-770 is currently approved for CF subjects with one copy of one of 38 mutations located throughout the CFTR sequence, including MSD1 (e.g. Di 10H, E193K), MSD2 (A1067T, R1070W), NBD1 (G551D, D579G), NBD2 (G1244E, G1349D), and other regions (E56K, P67L in the lasso domain) (2, 21, 22). Based on in silico docking and mutagenesis studies, two potential binding sites were identified for the Class I compounds VX-770 and GLPG1837 (23). The putative binding sites are located at the interface of the CFTR transmembrane domains involving residues D924, N1138 and S1141, or residues F229, F236, Y304, F312 and F931 (23). Recently, cryo-electron microscopy confirmed that VX-770 and GLPG1837 bind at the same site within the protein-lipid interface in a pocket formed by transmembrane helices 4, 5 and 8 (24). Evaluation of potential binding sites by alanine substitution revealed that a network of residues (including F236, Y304, F312 and F931, as well as L233, F305 and S308) interact directly with both VX-770 and GLPG1837 (24). Using pharmacological approaches, evidence was found that several previously reported potentiators including P2, P3 and P5 also bind at or near the VX-770 and GLPG1837 binding site. Given the broad efficacy of VX-770 for mutations throughout the CFTR protein, and the absence of large difference in CFTR structure with vs. without bound potentiator, Class I compounds such as VX-770 thermodynamically alter the closed-open equilibrium of CFTR (24).

Accordingly, exemplary Class I potentiators include, but are not limited to VX-770, P2, P3, P5, and GLPG1837. The structures of Class I potentiators are shown below (see also FIG. 1A):

VX-770 has the following structure:

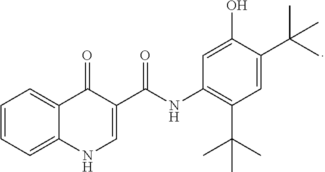 [SMILES: CC(C)(C)c1cc(C(C)(C)C)c(NC(=O)c2c[nH]c3ccccc3c2=O)cc1O]

P2 has the following structure:

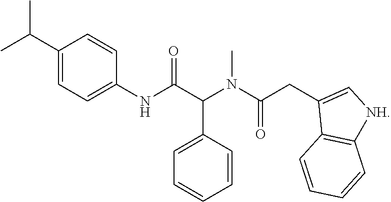 [SMILES: CC(C)c1ccc(NC(=O)C(c2ccccc2)N(C)C(=O)Cc2c[nH]c3ccccc23)cc1]

P3 has the following structure:

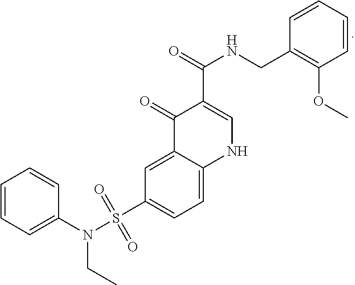 [SMILES: CCN(c1ccccc1)S(=O)(=O)c1ccc2[nH]cc(C(=O)NCc3ccccc3OC)c(=O)c2c1]

P5 has the following structure:

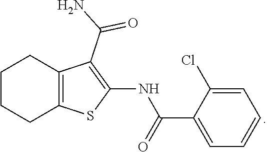 [SMILES: NC(=O)c1c(NC(=O)c2ccccc2Cl)sc2c1CCCC2]

GLPG1837 has the following structure:

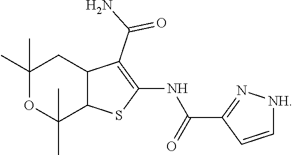

### Identifying Novel Class II Potentiators

Class II potentiators may bind to CFTR in a manner that stabilizes partial or misfolded NBD2 structurally or thermodynamically. When used as a co-potentiator with a Class I potentiator, a Class II compound is capable of acting in synergy to increase channel activity or otherwise rescue gating defects in CFTR mutants.

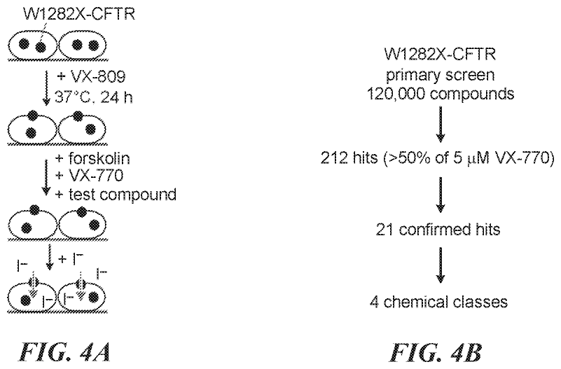

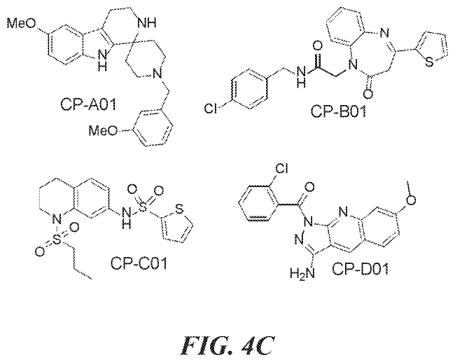

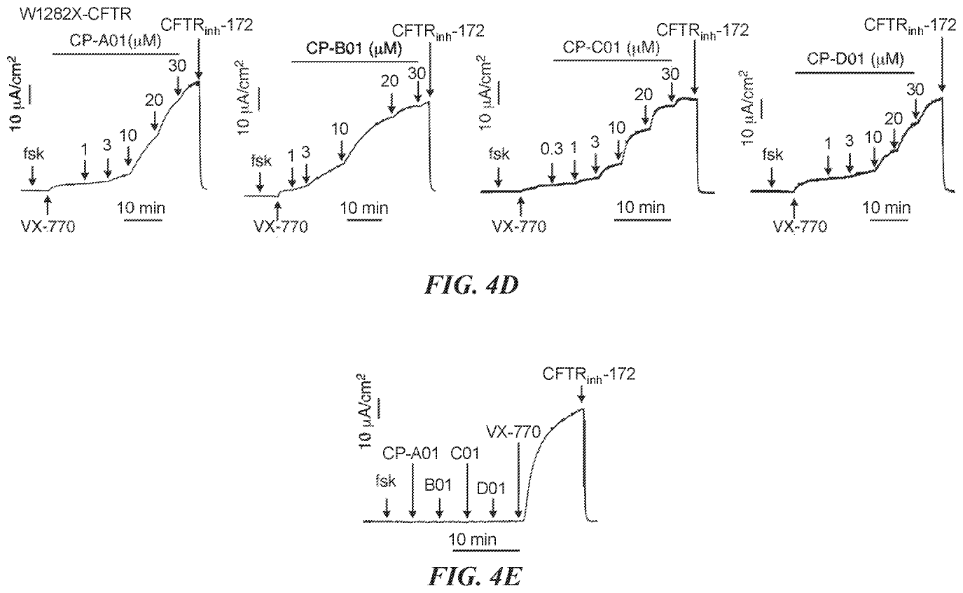

Given the utility of co-potentiators as possible CF therapeutics for several minimal function CFTR mutants, a screen was done to identify novel co-potentiator scaffolds. Screening used FRT cells stably expressing W1282X-CFTR and the halide-sensitive EYFP-H148Q/I152L/F46L (YFP) that were treated for 24 hours with 3 μM VX-809 to increase CFTR1281 cell surface expression (FIG. 4A). Just prior to assay cells were treated for 10 min with test compounds at 25 μM together with 20 μM forskolin and 15 nM VX-770. CFTR channel activity was deduced from the initial rate of YFP fluorescence quenching in response to addition of iodide-substituted phosphate buffered saline. Primary screening of 120,000 drug-like synthetic small molecules identified 212 compounds giving channel activity>50% of that produced by forskolin, VX-770 and 20 μM ASP-11. After initial confirmation with plate reader assays, short-circuit current measurement revealed 21 active compounds of four chemical classes, which were further studied.

The most active compounds included spiro[piperidine-4,1pyrido[3,4-b]indole] (CP-A01), phenylazepine (CP-B01), tetrahydroquinoline (CP-C01) and pyrazoloquinoline (CP-D01) (see FIG. 4C). Concentration-dependence measurements in VX-809-corrected W1282X-CF TR-expressing FRT cells in the presence of forskolin and 5 μM VX-770 demonstrated an ˜6-fold increase in VX-770 current with EC₅₀ of 10 μM, 5 μM, 8 μM and 15 μM for CP-A01, B-01, C01 and D01, respectively (FIG. 4D). Each of these compounds added to VX-809-corrected FRT cells expressing W1282X-CFTR produced little current without VX-770 (FIG. 4E). As found for ASP-11 (9, 10), synergy of W1282X-CFTR activation by VX-770 and the new Class II potentiators did not depend on the order of compound addition (data not shown).

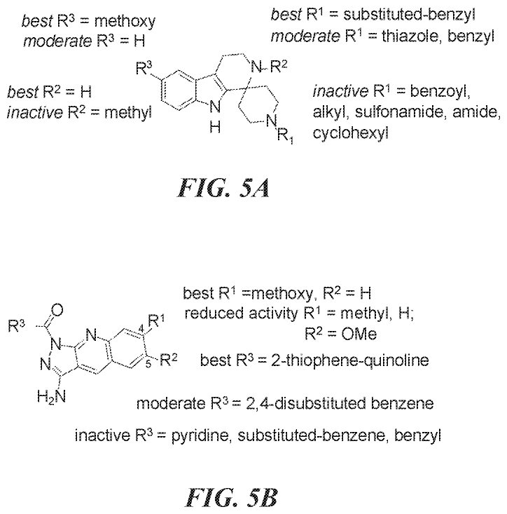

To establish structure-activity relationships, 240 commercially available spiro[piperidine-4,1pyrido[3,4-b]indoles]analogs and 160 pyrazoloquinoline analogs were tested in FRT cells expressing W 1282X-CFTR. FIG. 5A summarizes the structural determinants for activity for the spiro[piperidine-4,1pyrido[3,4-b]indoles] reported in Table 1A (CP-Axxx designations).

TABLE 1A
STRUCTURE-ACTIVITY DATA OF SELECTED SPIRO[PIPERIDINE-4,1PYRIDO[3,4-
B]INDOLES]ANALOGS

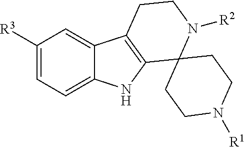

| | | | | | Relative |
|---|---|---|---|---|---|
| Compound | R^{l} | R² | R³ | EC₅₀ (μM)) | Vₘₐₓ %)* |
| A01 | 3-methoxy-benzyl | H | OMe | 10 | 100 |
| A061 | 2,4-difluoro-benzyl | H | OMe | 2.0 | 100 |
| A662 | 3,4-difluoro-benzyl | H | OMe | 2.6 | 100 |
| A666 | 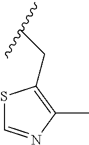 [SMILES: *Cc1scnc1C] | H | OMe | 5.1 | 91 |
| A357 | benzyl | H | H | 16 | 31 |
| A534 | 3-methoxy-benzyl | H | H | 13 | 24 |
| A600 | 3-methoxy-benzyl | Me | H | >30 | 4 |
| A714 | cyclohexyl | H | OMe | >30 | 18 |
| A815 | 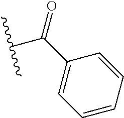 [SMILES: *C(=O)c1ccccc1] | H | H | >30 | −4 |
| A350 | 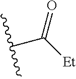 [SMILES: *C(=O)CC] | H | OMe | >30 | 7 |
| A956 | 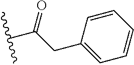 [SMILES: *C(=O)Cc1ccccc1] | H | OMe | >30 | 6 |
| A145 | SO₂—Me | H | OMe | >30 | 7 |
| A764 | Me | H | OMe | >30 | 6 |

*100% Vₘₐₓ corresponds to 20 μM ASP-11

In general, as shown in Table 1A, the methoxy substituent on the 4-position (R³) on the pyridoindole ring increased potency (compare A01 vs A534). N-methylation on the pyridoindole ring abolished activity (compare A534 vs A600). For substituent R1 on the piperidine ring, substituted benzyl gave greatest activity (A061 and A662). Other R1 substituents, including sulfonamide (A145), alkyl (A764) and carbocyclic (A714), reduced activity. Changing the benzylic carbon from methylene (CH2) to ketone (C═O) abolished activity (A815, A350 and A956). A061 with R¹ being 2,4-difluoro-benzyl was the most potent analog.

Based on the structure activity data, novel spiro[piperidine-4,1pyrido[3,4-b]indoles] compounds were synthesized according to the following Synthetic Scheme (I) and screened for their activities.

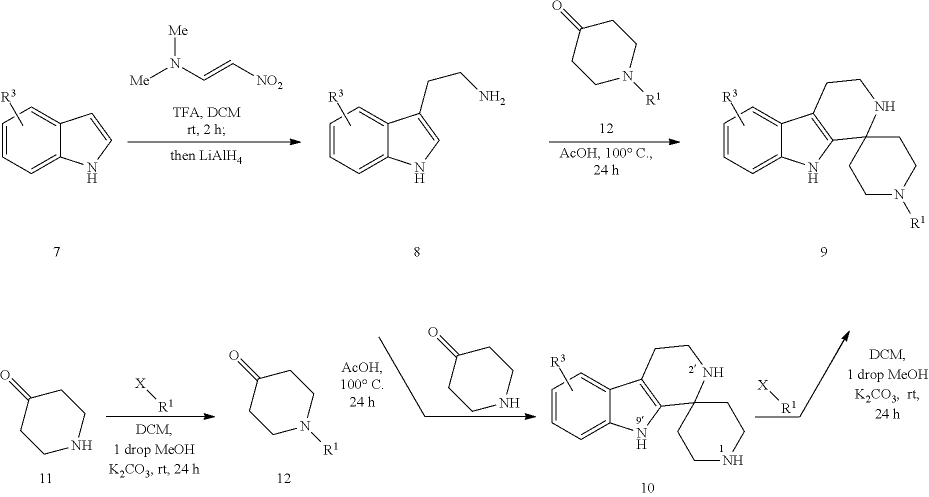

Table 1B summarizes certain novel spiro[piperidine-4,1pyrido[3,4-b]indoles] compounds suitable as co-potentiators with 2-17 folds of improvement.

TABLE 1B

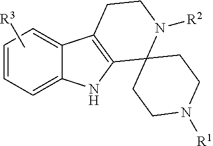

| | | | | | Relative |
|---|---|---|---|---|---|
| Compound | R¹ | R² | R³ | EC₅₀ (μM) | Vₘₐₓ (%)* |
| 1j | 3-chloro-2,4-difluoro- | H | 6-OMe | 4.5 | 120 |
| | -benzyl | | | | |
| 2d | 3,4,5-trifluoro-benzyl | H | 6-OMe | 2.6 | 121 |
| 2e | perfluoro-benzyl | H | 6-OMe | 2.1 | 134 |
| 2g | 2,3,4-trifluoro-benzyl | H | 6-OMe | 4.1 | 100 |
| 2i | 2,4,5-trifluoro-benzyl | H | 6-OMe | 0.6 | 97 |
| 5c | 2,4-difluoro-benzyl | H | 6-C1 | 2.5 | 89 |
| 5d | 2,4-difluoro-benzyl | H | 7-OMe | 2.4 | 100 |

FIG. 5B summarizes the structural determinants for the pyrazoloquinolines

TABLE 2
STRUCTURE-ACTIVITY DATA OF SELECTED PYRAZOLOQUINOLINE ANALOGS

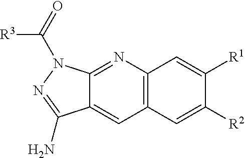

| | | | | | Relative |
|---|---|---|---|---|---|
| Compound | R¹ | R² | R³ | EC₅₀ (μM) | Vₘₐₓ (%)* |
| D01 | OMe | H | 2-chlorobenzene | 15 | 90 |
| D003 | OMe | H | 4-pyridine | >30 | 1 |
| D010 | OMe | H | 4-fluorobenzene | 3.6 | 24 |
| D012 | OMe | H | 3,4,5-trimethoxybenzene | >30 | 3 |
| D018 | OMe | H | 2-chloro-4-fluoro-benzene | 2.4 | 92 |
| D025 | OMe | H | 3-methyl-4-nitro-benzene | >30 | 3 |
| D035 | OMe | H | 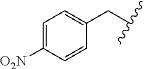 [SMILES: *Cc1ccc([N+](=O)[O-])cc1] | >30 | 1 |
| D036 | OMe | H | 3-nitro-4-chloro-benzene | >30 | 11 |
| D038 | OMe | H | 2-chloro-4-nitro-benzene | 1.7 | 100 |
| D086 | OMe | H | 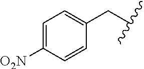 [SMILES: *Cc1ccc([N+](=O)[O-])cc1] | >30 | 3 |
| D123 | OMe | H | 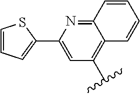 [SMILES: *c1cc(-c2cccs2)nc2ccccc12] | 0.3 | 100 |
| D136 | Me | H | 4-fluorobenzene | 18 | 11 |
| D138 | H | OMe | 2-chloro-4-nitro-benzene | 3.4 | 21 |

*100% Vₘₐₓ corresponds to 20 μM ASP-11

As shown in Table 2, the position of the methoxy on the quinoline ring affected activity as changing from the 4th to 5th position greatly reduced potency (D038 vs D138). Replacing the electron-donating methoxy group to electron-neutral methyl group also reduced activity (D010 vs D136). For R3, pyridine (D003), benzyl (D035) and substituted methyl-pyrazole (D086) abolished activity. Substituted benzenes had a range of potencies with 2,4-disubstituted compounds including 2-chloro-4-fluorobenzene (D018) and 2-chloro-4-nitrobenzene (D038) being the most potent. The D123 pyrazoloquinoline with R3 substituted with thiophene-quinoline heterocycle gave the best potency.

Short-circuit current measurements were done for the most potent spiro[piperidine-4,1pyrido[3,4-b]indole] (CP-A061) and pyrazoloquinoline (CP-D123). FIGS. 5C and 5D show data in N1303K- and Q1313X-expressing FRT cells. Following forskolin and VX-770, concentration-dependent increases in current were seen following addition of co-potentiators, with current fully inhibited by CFTRᵢₙₕ-172. The calculated EC₅₀ values were 2.9 μM and 300 nM for CP-A061 and CP-D123, respectively. Similar EC₅₀ values of 5 μM and 320 nM for CP-A061 and CP-D123 were found in FRT cells expressing Q1313X-CFTR.

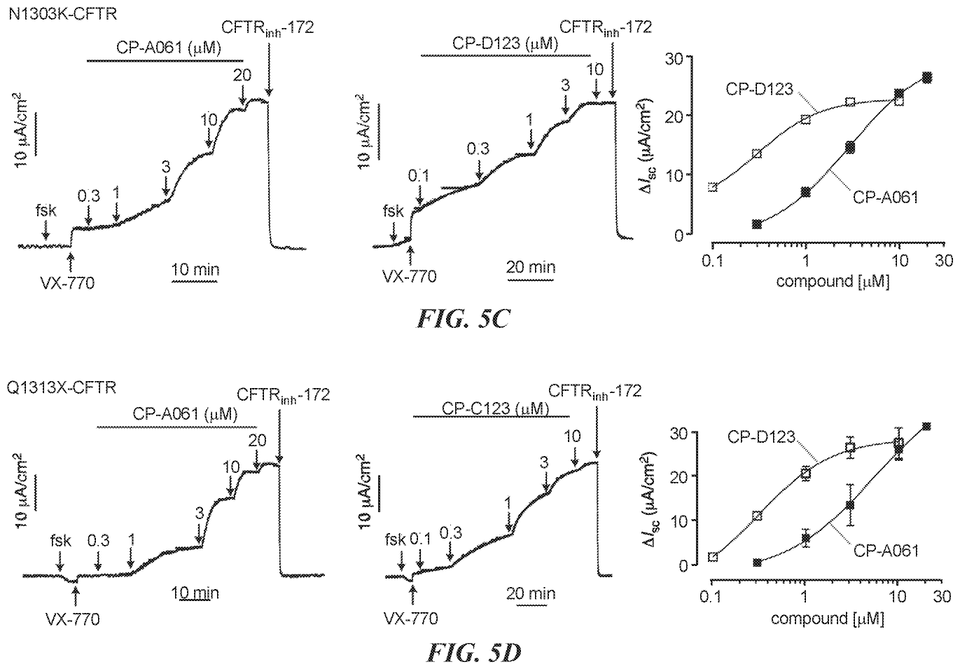

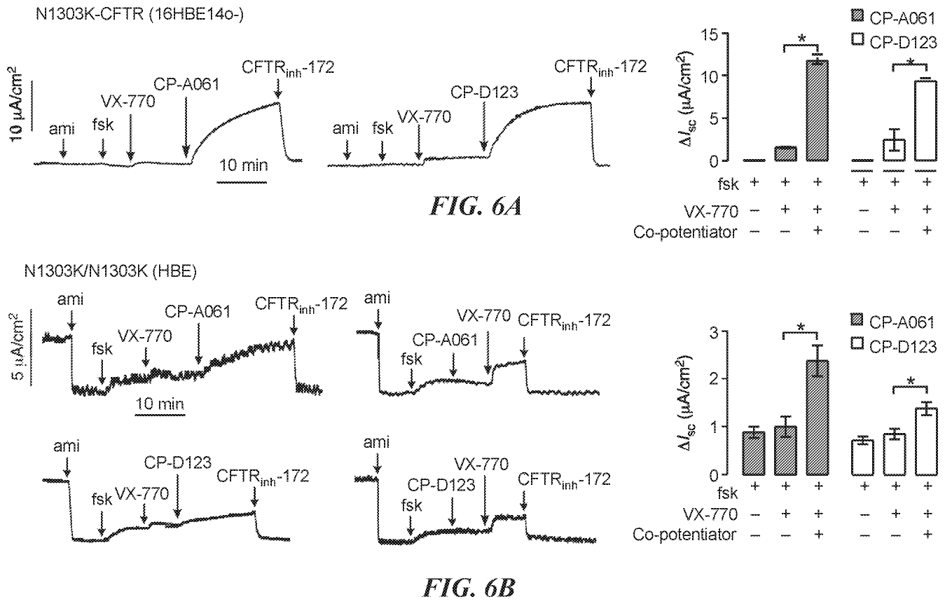

To test the efficacy of new co-potentiators in human airway cell models, short-circuit current was measured in 16HBE14o-human airway epithelial cells in which the endogenous CFTR gene was edited to contain the N1303K mutation (16HBE-N1303Kge, (20)) and in primary cultures of human bronchial epithelial cells from a N1303K homozygous CF subject. Addition of forskolin and then VX-770 to 16HBE-N1303Kge cells gave a limited response (FIG. 6A). Subsequent addition of CP-A061 or CP-D123 produced CFTRᵢₙₕ-172-inhibitable responses of ˜10 μA/cm², ˜6-fold greater that that produced by VX-770 alone. Increased short-circuit current was also found for these co-potentiators in the primary human bronchial epithelial cell cultures (FIG. 6B).

Thus, according to certain embodiments, the Class II potentiators are spiro[piperidine-4,1pyrido[3,4-b]indole] derivatives represented by Structure (A):

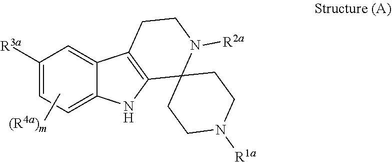

wherein:

m is 0, 1, 2 or 3;

R^{1a} is optionally substituted arylalkyl, optionally substituted heteroaryl or optionally substituted heteroarylalkyl;

R^{2a} is H or C₁-C₆ alkyl;

R^{3a} is H, halo or C₁-C₆ alkoxy; and

R^{4a} is H, C₁-C₆ alkoxy or C₁-C₆ alkyl.

In more specific embodiments, the Class II potentiators are Compounds A01, A061, A662, A666, A357 or A534 of Table 1A and Compounds 1j, 2d, 2e, 2g, 2i, 5c and 5d of Table 1B.

In other embodiments, the Class II potentiators are phenylazepine derivatives represented by Structure (B):

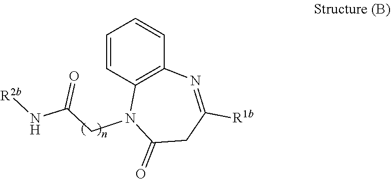

wherein:

n is 1 or 2;

R^{1b} is a 5- or 6-membered heteroaryl; and

R^{2b} is an optionally substituted arylalkyl.

In a more specific embodiment, the Class II potentiator is Compound CP-B01 (of FIG. 4C).

In certain other embodiments, the Class II potentiators are tetrahydroquinoline derivatives represented by Structure (C):

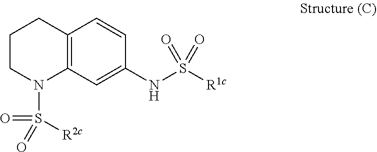

wherein:

R^{1c} is a 5- or 6-membered heteroaryl; and

R^{2c} is C₁-C₆ alkyl.

In a more specific embodiment, the Class II potentiator is Compound CP-C01 (of FIG. 4C).

In yet other embodiments, the Class II potentiators are pyrazoloquinoline derivatives represented by Structure (D):

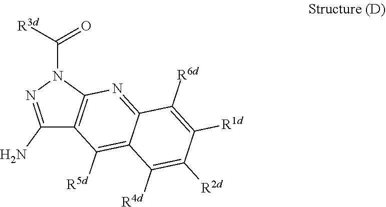

wherein:

R^{1d} is H, C₁-C₆ alkyl, or C₁-C₆ alkoxy;

R^{2d} is H or C₁-C₆ alkoxy;

R^{3d} is substituted aryl, or substituted heteroaryl; and

R^{4d}, R^{5d}, and R^{6d} are independently H, C₁-C₆ alkyl, or C₁-C₆ alkoxy.

In a more specific embodiment, the Class II potentiator is Compound D01, D018, D038 or D123 of Table 2.

### Co-Potentiator Therapy for Treating Certain CF Subjects

The co-potentiator scaffolds described herein are shown to have nanomolar potency that, in synergy with Class I potentiators such as VX-770, are capable of activating CFTRs with NBD2 mutations including N1303K-CFTR. Thus, potentiator/co-potentiator combination therapy may be effective in a subset of minimal function missense, nonsense and deletion mutations in CFTR that cause cystic fibrosis and are not responsive to current CFTR modulator combinations.

In particular, CF subjects of a variety of missense, nonsense and deletion mutations in NBD2, including N1303K- and I1234del-CFTR, can benefit from two distinct potentiators. In prior studies on the responses of >50 rare CFTR missense mutations to VX-770 and VX-809, N1303K-CFTR was not responsive to VX-770 and showed very limited response to VX-809 (25). This is consistent with the notion that the N1303K mutation causes defective CFTR folding, regulation and gating (26). Han et al. (2018) reported diverse responses to CFTR modulators—some mutations (P5L, G27R, S492F, Y1032C) responded to VX-809 but not VX-770, some (M348V) to VX-770 but not VX-809, and some (G85E, R560T, A561E, Y563N) with no response. In contrast, robust activation of N1303K-CFTR is observed with co-potentiators in the absence of a corrector in a human airway epithelial cell lines expressing endogenous levels of gene-edited CFTR (FIG. 6A). In primary human bronchial cell cultures from a homozygous N1303K-CFTR subject, CP-A061 produced ˜2.5 μA/cm² of CFTRᵢₙₕ-172-inhibitable current (FIG. 6B). Based on measurements performed using similarly cultured non-CF and CF human airway epithelial cell cultures, we estimate that 2.5 μA/cm² of CFTR current is equivalent to ˜20% of wild type CFTR activity, which is potentially of clinical benefit. This percentage value would further increase with use of an effective corrector to increase N1303K-CFTR cell-surface CFTR expression.

It is difficult to estimate the number of CF subjects that might benefit from co-potentiator therapy. The N1303K allele is found in 2,147 subjects in the CFTR2 databases, of which 99 are homozygous and >400 would not be benefitted by VX-770 or therapies that targeting one F508del-CFTR allele. Similarly, c.3700 A>G is found in 28 subjects, of which 5 are homozygous. It is noted that many countries in which N1303K and c.3700 A>G are prevalent do not contribute to the CFTR2 database (27, 28). We previously showed that ASP-11 activates G551D-CFTR, as do the new co-potentiators identified here (not shown). The G551D allele is found in ˜3000 CF subjects in CFTR2, including 69 homozygous subjects. In addition, the co-potentiators were effective in increasing CFTR chloride current for several truncated forms of CFTR resulting from premature termination codons (PTCs) located in NBD2. PTCs result in nonsense-mediated degradation (NMD) of transcript resulting in reduced synthesis of truncated protein products (29, 30). CFTR transcript levels have been reported from ˜10-75% of levels in non-CF cells (31, 32), though one study reported complete absence of W1282X-CFTR transcript in cells from a single CF subject (33). Co-potentiators may thus be therapeutically beneficial for PTCs in NBD2, alone if sufficient transcript is present, or in combination with other drugs such as NMD inhibitors or read-through agents.

Provided herein is a method of treating cystic fibrosis in a CF subject having one or more CFTR missense, nonsense and deletion mutations, the method comprising administering a Class I potentiator and at least one Class II potentiator to the CF subject, wherein the Class II potentiator has one of the structures (A), (B), (C), or (D), as described herein.

In certain embodiments, the missense, nonsense, deletion, or truncation mutation is in NBD2, including for example, N1303K, G542X, W1282X, G551D, I1234del-CFTR, Q1313X, and c.3700 A>G. In certain more specific embodiments, the missense, nonsense, deletion, or truncation mutation is selected from the group consisting of N1303K, W1282X, and G551D. In certain specific embodiments, the missense, nonsense, deletion, or truncation mutation is N1303K.

In some embodiments, the Class I potentiator is selected from the group consisting of VX-770, P2, P3, P5, and GLPG1837.

In certain embodiment, the Class II potentiator is a spiro[piperidine-4,1pyrido[3,4-b]indole] derivative represented by Structure (A):

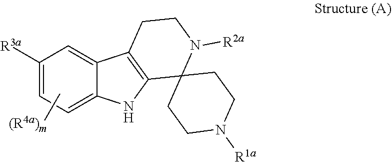

wherein:

m is 0, 1, 2 or 3;

R^{1a} is optionally substituted arylalkyl, optionally substituted heteroaryl or optionally substituted heteroarylalkyl;

R^{2a} is H or C₁-C₆ alkyl;

R^{3a} is H, halo or C₁-C₆ alkoxy; and

R^{4a} is H, C₁-C₆ alkoxy or C₁-C₆ alkyl.

In some more specific embodiments, R^{1a} is an optionally substituted arylalkyl (e.g., benzyl). In more specific embodiments, In some embodiments, R^{1a} is unsubstituted benzyl. In some embodiments, R^{1a} is a substituted benzyl. In some specific embodiments, R^{1a} is substituted with one or more substituents selected from the group consisting of halo, and C₁-C₆ alkoxy. In some embodiments, R^{1a} is substituted with one or more substituents selected from the group consisting of methoxy, and fluoro. In preferred embodiments, R^{1a} is benzyl in which the phenyl moiety is substituted with two or more halo (e.g., fluoro). In some embodiments, R^{1a} is selected from the group consisting of benzyl, 3-methoxy-benzyl, 2,4-difluoro-benzyl, 3,4-difluoro-benzyl, 3-chloro-2,4-difluoro-benzyl, 3,4,5-trifluoro-benzyl, perfluoro-benzyl, 2,3,4-trifluoro-benzyl, 2,4,5-trifluoro-benzyl.

In some embodiments, R^{1a} is an optionally substituted heteroaryl, including for example, optionally substituted thiazolyl.

In other embodiments, R^{1a} is an optionally substituted heteroarylalkyl. In some embodiments, R^{1a} is a heteroarylalkyl substituted with alkyl. In some embodiments, R^{1a} has the following structure:

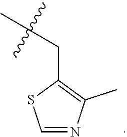 [SMILES: *Cc1scnc1C]

In certain embodiments, R^{2a} is H. In some embodiments, R^{2a} is C₁-C₆ alkyl (e.g., methyl, ethyl, propyl). In more specific embodiments, R^{2a} is methyl.

In some embodiments, R^{3a} is H. In some specific embodiments, R^{3a} is C₁-C₆ alkoxy (e.g., methoxy, ethoxy, propoxy) or halo (e.g., fluoro or chloro). In more specific embodiments, R^{3a} is methoxy or chloro.

In preferred embodiments, m is 0.

In other embodiments, m is 1, and R^{4a} is C₁-C₆ alkoxy (e.g., methoxy, ethoxy, propoxy).

In some embodiments, the compound of structure (A) has one of the following structures:

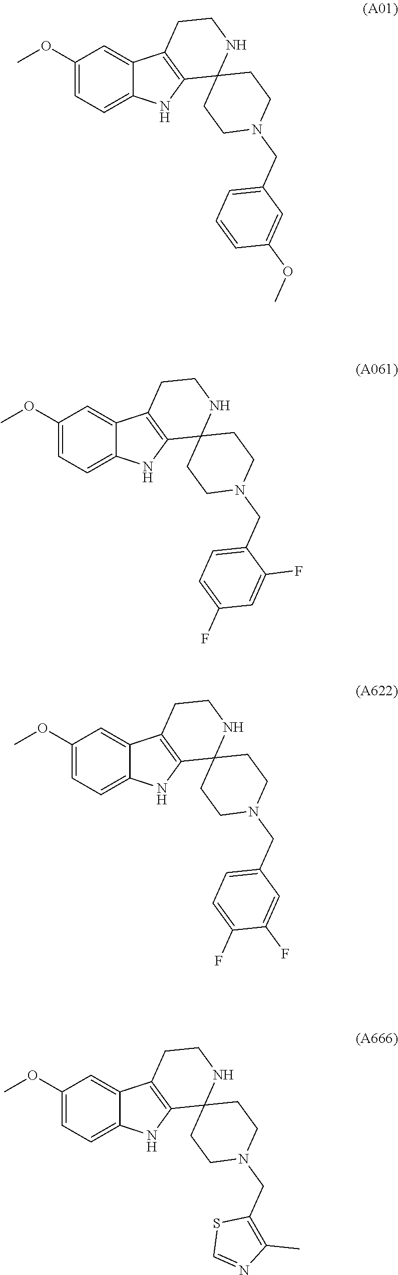

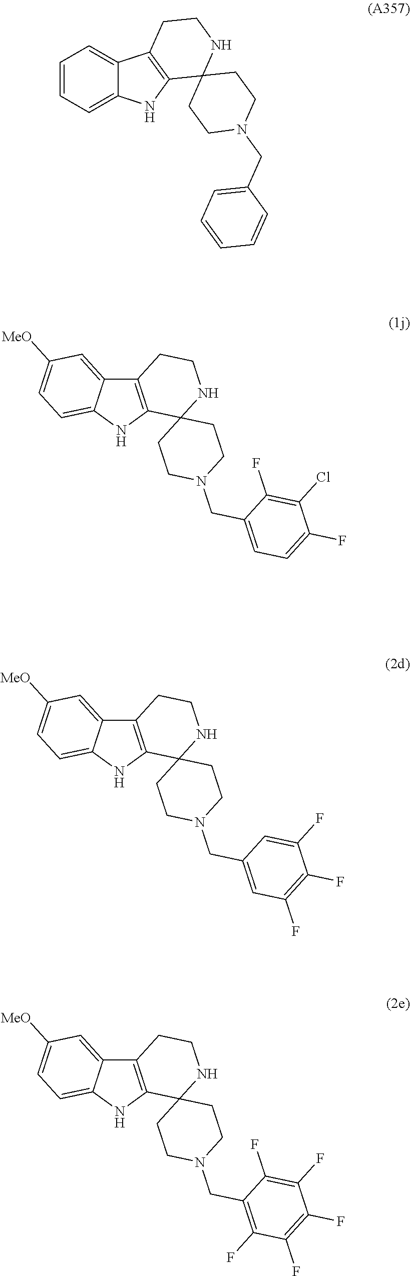 [SMILES: c1ccc(CN2CCC3(CC2)NCCc2c3[nH]c3ccccc23)cc1 ; COc1ccc2[nH]c3c(c2c1)CCNC31CCN(Cc2ccc(F)c(Cl)c2F)CC1 ; COc1ccc2[nH]c3c(c2c1)CCNC31CCN(Cc2cc(F)c(F)c(F)c2)CC1 ; COc1ccc2[nH]c3c(c2c1)CCNC31CCN(Cc2c(F)c(F)c(F)c(F)c2F)CC1]

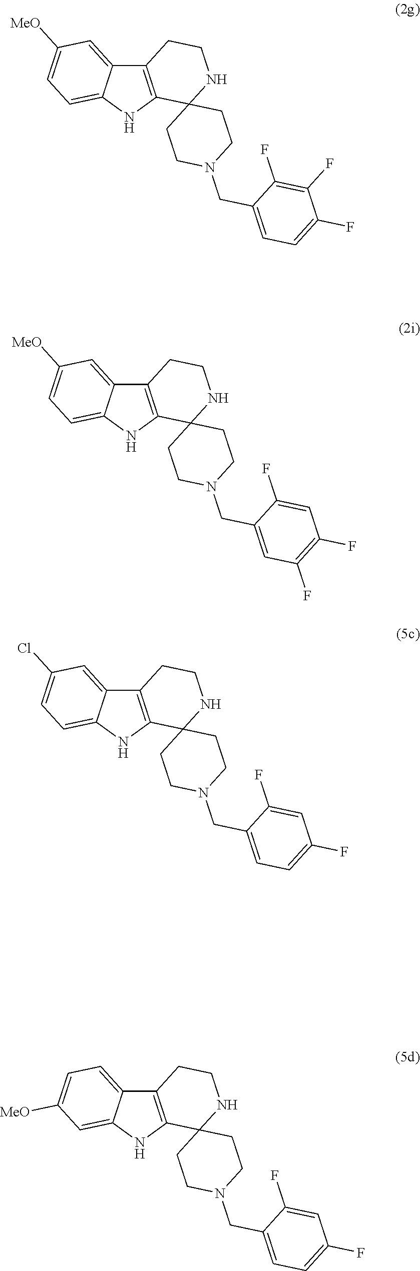 [SMILES: COc1ccc2[nH]c3c(c2c1)CCNC31CCN(Cc2ccc(F)c(F)c2F)CC1 ; COc1ccc2[nH]c3c(c2c1)CCNC31CCN(Cc2cc(F)c(F)cc2F)CC1 ; Fc1ccc(CN2CCC3(CC2)NCCc2c3[nH]c3ccc(Cl)cc23)c(F)c1 ; COc1ccc2c3c([nH]c2c1)C1(CCN(Cc2ccc(F)cc2F)CC1)NCC3]

In some embodiments, the Class II potentiator has the following structure (B):

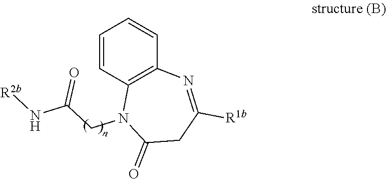

wherein:

n is 1 or 2;

R^{1b} is a 5- or 6-membered heteroaryl; and

R^{2b} is an optionally substituted arylalkyl.

In some specific embodiments, R^{1b} is a 5-membered heteroaryl. In certain embodiments, R^{1b} is unsubstituted. In some specific embodiments, R^{1b} comprises sulfur.

In certain embodiments, R^{1b} has the following structure:

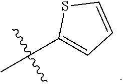 [SMILES: *c1cccs1]

In some embodiments, R^{2b} benzyl. In certain embodiments, R^{2b} is substituted. In more specific embodiments, R^{2b} is substituted with chloro. In some embodiments, R² has the following structure:

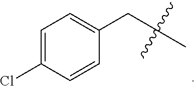 [SMILES: *Cc1ccc(Cl)cc1]

In some embodiments, n is 1.

In some specific embodiments, the Class II potentiator has the following structure:

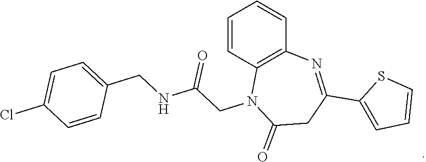 [SMILES: O=C(CN1C(=O)CC(c2cccs2)=Nc2ccccc21)NCc1ccc(Cl)cc1]

In some embodiments, the Class II potentiator has the following structure (C):

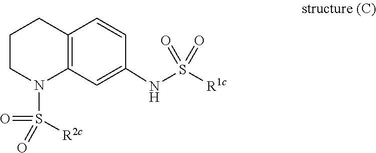

wherein:

R^{1c} is a 5- or 6-membered heteroaryl; and

R^{2c} is C₁-C₆ alkyl.

In certain embodiments, R^{1c} is a 5-membered heteroaryl. In other embodiments, R^{1c} is unsubstituted. In some specific embodiments, R^{1c} comprises sulfur. In some embodiments, R^{1c} has the following structure:

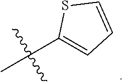 [SMILES: *c1cccs1]

In more specific embodiments, R^{2c} is n-propyl.

In some specific embodiments, the Class II potentiator has the following structure:

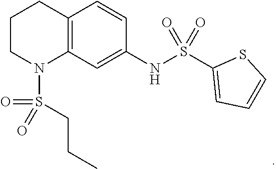 [SMILES: CCCS(=O)(=O)N1CCCc2ccc(NS(=O)(=O)c3cccs3)cc21]

In yet other embodiments, the Class II potentiator is a pyrazoloquinoline derivative represented by Structure (D):

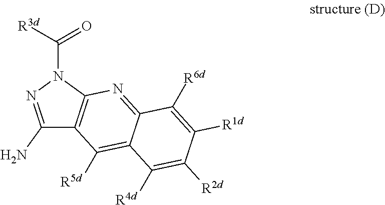

wherein:

R^{1d} is H, C₁-C₆ alkyl, or C₁-C₆ alkoxy;

R^{2d} is H or C₁-C₆ alkoxy;

R^{3d} is substituted aryl, substituted heteroaryl; and

R^{4d}, R^{5d}, and R^{6d} are independently H, C₁-C₆ alkyl, or C₁-C₆ alkoxy.

In some embodiments, R^{1d} is H. In some embodiments, R^{1d} is C₁-C₆ alkyl (e.g., methyl, ethyl, propyl). In some more specific embodiments, R^{1d} is methyl. In preferred embodiments, R^{1d} is C₁-C₆ alkoxy (e.g., methoxy, ethoxy, propoxy). In more specific embodiments, R^{1d} is methoxy.

In certain embodiments, R^{2d} is H. In other embodiments, R^{2d} is C₁-C₆ alkoxy (e.g., methoxy, ethoxy, propoxy). In more specific embodiments, R^{2d} is methoxy.

In some embodiments, R^{3d} is substituted aryl. In some specific embodiments, R^{3d} is substituted phenyl having one or more substituents selected from the group consisting of halo, C₁-C₆ alkoxy, C₁-C₆ alkyl, nitro, and heteroaryl. In some embodiments, R^{3d} is selected from the group consisting of 2-chlorobenzene, 4-fluorobenzene, 3,4,5-trimethoxybenzene, 2-chloro-4-fluoro-benzene, 3-methyl-4-nitro-benzene, 3-nitro-4-chloro-benzene, 2-chloro-4-nitro-benzene, 4-fluorobenzene, and 2-chloro-4-nitro-benzene.

In some embodiments, R^{3d} is substituted heteroaryl. In further amendments, R^{3d} is substituted bicyclic heteroaryl. In more specific embodiments, R^{3d} is a substituted quinolinyl. In other more specific embodiments, R^{3d} is a substituted thienyl. In some embodiments, R^{3d} is a substituted quinolinyl (e.g., substituted with one or more substituents selected from the group consisting of halo, C₁-C₆ alkoxy, C₁-C₆ alkyl, nitro, and heteroaryl). In more specific embodiments, R^{3d} is quinolinyl substituted with one or more substituents selected from the group consisting of chloro, fluoro, methoxy, methyl, nitro, and thiophenyl. In more specific embodiments, R^{3d} is quinolinyl substituted with thiophenyl. In some embodiments, R^{3d} has the following structure:

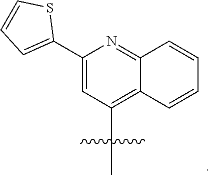 [SMILES: *c1cc(-c2cccs2)nc2ccccc12]

In some embodiments, the compound of structure (D) has one of the following structures:

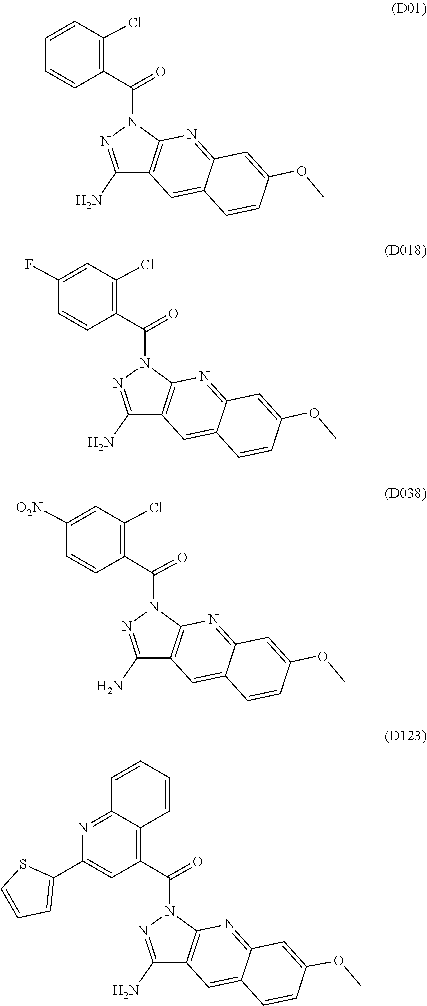 [SMILES: COc1ccc2cc3c(N)nn(C(=O)c4ccccc4Cl)c3nc2c1 ; COc1ccc2cc3c(N)nn(C(=O)c4ccc(F)cc4Cl)c3nc2c1 ; COc1ccc2cc3c(N)nn(C(=O)c4ccc([N+](=O)[O-])cc4Cl)c3nc2c1 ; COc1ccc2cc3c(N)nn(C(=O)c4cc(-c5cccs5)nc5ccccc45)c3nc2c1]

### Concomitant Treatment of CFTR Co-Potentiators and One or More CFTR Modulators

In some embodiments, the CFTR co-potentiator therapy disclosed herein can be combined with one or more additional CFTR modulators. Thus, one embodiment provides a method for treating cystic fibrosis in a CF subject having one or more CFTR missense, nonsense and deletion mutations, the method comprising: administering a Class I potentiator and at least one Class II potentiator to the CF subject, wherein the Class II potentiator has one of the Structures (A), (B), (C), or (D), and administering to the CF subject with a further CFTR modulator.

In more specific embodiments, the one or more additional CFTR modulator may be a corrector, an amplifier, a read-through agent, or a combination thereof. As used herein, a CFTR corrector refers to a compound that increases the amount of functional CFTR protein to the cell surface, resulting in enhanced ion transport. Examples of suitable correctors include, without limitation, VX-809, VX-661, VX-983, VX-152, VX-440, VX-445, VX-659, GLPG2222, GLPG3221, GLPG2737, GLPG2851 and/or GLPG2665, PTI-801.

In other embodiments, the additional CFTR modulator is an amplifier, which acts to increase the amount of CFTR protein produced by the cells. With increased production of CFTR protein, potentiators and/or correctors would be able to allow even more chloride to flow across the cell membrane. Examples of suitable amplifier include, without limitation, PTI-428. Additional examples may be found in WO2017/223188, which reference is incorporated herein by reference in its entirety.

In further embodiments, the additional CFTR modulator is a read-through agent, which promotes read-through of PTCs to generate full-length CFTR. Examples of suitable amplifier include, without limitation, ataluren (PTC124).

There is no particular order by which the co-potentiators and the additional CFTR modulators are administered. They can be administers simultaneously (e.g., all agents are combined in a single unit dosage form) or separately.

### Chemistry Definitions

“Alkyl” means a straight chain or branched, noncyclic, unsaturated or partially unsaturated aliphatic hydrocarbon containing from 1 to 12 carbon atoms. A lower alkyl refers to an alkyl that has any number of carbon atoms between 1 and 6 (i.e., C₁-C₆ alkyl) Representative saturated straight chain alkyls include methyl, ethyl, n-propyl, n-butyl, n-pentyl, n-hexyl, and the like, while saturated branched alkyls include isopropyl, sec-butyl, isobutyl, tert-butyl, tert-pentyl, heptyl, n-octyl, isopentyl, 2-ethylhexyl and the like. Alkyl may be optionally substituted by one or more substituents as defined herein.

“Alkoxy” refers to the radical of —O-alkyl. Examples of alkoxy include methoxy, ethoxy, and the like. The alkyl moiety of alkoxy may be optionally substituted by one or more substituents as defined herein.

“Aryl” means an aromatic carbocyclic moiety such as phenyl or naphthyl (i.e., naphthalenyl) (1- or 2-naphthyl) or anthracenyl (e.g., 2-anthracenyl).

“Arylalkyl” (e.g., phenylalkyl) means an alkyl having at least one alkyl hydrogen atom replaced with an aryl moiety, such as —CH₂-phenyl (i.e., benzyl), —CH═CH-phenyl, —C(CH₃)═CH-phenyl, and the like.

“Heteroaryl” refers to a 5- to 14-membered ring system comprising one to thirteen carbon atoms, one to six heteroatoms selected from the group consisting of nitrogen, oxygen and sulfur, and at least one aromatic ring. For purposes of certain embodiments of this disclosure, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, benzoxazolinonyl, benzimidazolthionyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, pteridinonyl, purinyl, pyrrolyl, pyrazolyl, pyridinyl, pyridinonyl, pyrazinyl, pyrimidinyl, pryrimidinonyl, pyridazinyl, pyrrolyl, pyrido[2,3-d]pyrimidinonyl, quinazolinyl, quinazolinonyl, quinoxalinyl, quinoxalinonyl, quinolinyl, isoquinolinyl, tetrahydroquinolinyl, thiazolyl, thiadiazolyl, thieno[3,2-d]pyrimidin-4-onyl, thieno[2,3-d]pyrimidin-4-onyl, triazolyl, tetrazolyl, triazinyl, and thiophenyl (i.e. thienyl). Unless stated otherwise specifically in the specification, a heteroaryl group is optionally substituted.

“Heteroarylalkyl” (e.g., pyrazolylalkyl) means an alkyl having at least one alkyl hydrogen atom replaced with an heteroaryl moiety, such as —CH₂-pyrazolyl, —CH₂-pyridinyl, —CH₂-quinolinyl and the like.

“Halogen” or “halo” means fluoro, chloro, bromo, and iodo.

All the above groups may be “optionally substituted,” i.e., either substituted or unsubstituted. The term “substituted” as used herein means any of the above groups (i.e., alkyl, alkoxy, alkoxyalkyl, aryl, aralkyl, heterocyclyl, heterocyclylalkyl, heteroaryl, heteroarylalkyl and/or trifluoroalkyl), may be further functionalized wherein at least one hydrogen atom is replaced by a bond to a non-hydrogen atom substituent. Unless stated specifically in the specification, a substituted group may include one or more substituents selected from: ═O, —CO₂H, nitrile, nitro, —CONH₂, hydroxyl, thiooxy, alkyl, alkylene, alkoxy, alkoxyalkyl, alkylcarbonyl, alkyloxycarbonyl, aryl, aralkyl, arylcarbonyl, aryloxycarbonyl, aralkylcarbonyl, aralkyloxycarbonyl, aryloxy, cycloalkyl, cycloalkylalkyl, cycloalkylcarbonyl, cycloalkylalkylcarbonyl, cycloalkyloxycarbonyl, heterocyclyl, heteroaryl, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, thioalkyl triarylsilyl groups, perfluoroalkyl or perfluoroalkoxy, for example, trifluoromethyl or trifluoromethoxy.

### Pharmaceutical Compositions

A pharmaceutical composition, as used herein, refers to a mixture of a compound described herein (e.g., co-potentiators) with other chemical components, such as carriers, stabilizers, diluents, dispersing agents, suspending agents, thickening agents, and/or excipients.

In certain embodiments, compounds described herein are formulated for oral administration. Compounds described herein are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

Useful pharmaceutical compositions also, optionally, include solubilizing agents to aid in the solubility of a compound described herein. The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, useful pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, useful compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some embodiments, the compounds described herein are formulated or administered in conjunction with liquid or solid tissue barriers also known as lubricants. Examples of tissue barriers include, but are not limited to, polysaccharides, polyglycans, seprafilm, interceed and hyaluronic acid.

The co-potentiators described herein can be administered simultaneously or separately. This administration in combination can include simultaneous administration of the two agents in the same dosage form, simultaneous administration in separate dosage forms, and separate administration. That is, a compound described herein and any of the agents described above can be formulated together in the same dosage form and administered simultaneously. Alternatively, a compound of the invention and any of the agents described above can be simultaneously administered, wherein both the agents are present in separate formulations. In another alternative, a compound of the present invention can be administered just followed by and any of the agents described above, or vice versa. In some embodiments of the separate administration protocol, a compound of the invention and any of the agents described above are administered a few minutes apart, or a few hours apart, or a few days apart.

### EXAMPLES

### Abbreviations

ASP-11: 1-butyl-N-(4-ethylphenyl)-1H-pyrrolo[2,3-b]pyridine-3-sulfonamide

CFTR: cystic fibrosis transmembrane conductance regulator

EC₅₀: half-maximal effective concentration

FRT: Fisher rat thyroid

GLPG1837: N-(3-carbamoyl-5,5,7,7-tetramethyl-5,7-dihydro-4H-thieno[2,3-c]pyran-2-yl)-1H-pyrazole-5-carboxamide

MSD: membrane spanning domain

NBD: nucleotide binding domain

P2: N-methyl-N-[2-[[4-(1-methylethyl)phenyl]amino]-2-oxo-1-phenylethyl]-1H-indole-3-acetamide

P3: 6-(ethyl-phenyl-sulfonyl)-4-oxo-1,4-dihydro-quinoline-3-carboxylic acid 2-methoxy-benzylamide

P5: 2-(2-chloro-benzylamino)-4,5,6,7-tetrahydro-benzo[b]thiophene-3-carboxylic acid amide

VX-770: N-(2,4-ditert-butyl-5-hydroxyphenyl)-4-oxo-1H-quinoline-3-carboxamide

VX-809: 3-[6-[[1-(2,2-difluoro-1,3-benzodioxol-5-yl)cyclopropanecarbonyl]amino]-3-methylpyridin-2-yl]benzoic acid

VX-661: 1-(2,2-difluoro-1,3-benzodioxol-5-yl)-N-[1-[(2R)-2,3-dihydroxypropyl]-6-fluoro-2-(1-hydroxy-2-methylpropan-2-yl)indol-5-yl]cyclopropane-1-carboxamide

YFP: yellow fluorescent protein

### General Experimental

### Materials and Methods:

### Chemicals

All compounds described in this manuscript have >95% purity. The analytical method used to determine purity was 1H NMR (see the accompanying Supporting Information file which provides the 1H- and ¹³C-NMR for the thirty-seven compounds assayed) and HPLC/HRMS. For HRMS analysis, samples were analyzed by flow-injection analysis into a Thermo Fisher Scientific LTQ Orbitrap (San Jose, Calif.) operated in the centroided mode. Samples were injected into a mixture of 50% MeOH/H2O and 0.1% formic acid at a flow of 0.2 mL/min. Source parameters were 5.5 kV spray voltage, capillary temperature of 275° C. and sheath gas setting of 20. Spectral data were acquired at a resolution setting of 100,000 FWHM with the lockmass feature, which typically results in a mass accuracy<2 ppm.

VX-809, VX-770, GLPG1837 and CFTRᵢₙₕ-172 were purchased from Selleck Chemicals (Boston, Mass.). Potentiators P2 (PG-01; Pedemonte, N. et al., (2005) Phenylglycine and sulfonamide correctors of defective delta F508 and G551D cystic fibrosis transmembrane conductance regulator chloride-channel gating. Mol. Pharmacol. 67, 1979-1807), P3 (SF-03; Pedemonte, 2005) and P5 (dF508_{act}-02; Yang, H. et al., (2003) Nanomolar-affinity small-molecular potentiators of DF508-CFTR chloride channel gating. J. Biol. Chem. 278, 35079-35085) were obtained from an in-house repository of CFTR modulators. For screening, 120,000 diverse drug-like synthetic compounds (i.e., Structures (A), (B), (C), and (D); ChemDiv Inc., San Diego, Calif.) were tested. Other chemicals were purchased from Sigma unless otherwise stated.

### Complementary DNA Constructs

Complementary DNAs (cDNAs) for the I1234del-, W1282C/L/R and Q1313X-mutants CFTRs were generated using standard techniques. In brief, gBLOCK gene fragment (Integrated DNA Technology, Coralville, Iowa) were synthesized and introduced into full-length CFTR cDNA in the vector pcDNA3.1/Zeo (+) (Invitrogen). For subcloning, I1234del-CFTR was generated using a HindIII site at position 3171-3176 of the CFTR cDNA; for W1282C/L/R and Q1313X-CFTR a BstXI site at position 3801-3812 of CFTR cDNA was used. The mutated CFTR cDNAs were subcloned into vector pIRESpuro3 (Clontech, Mountain View, Calif.) using NheI and NotI restriction sites. All constructs were confirmed by sequencing.

### Cell Culture Models

Fischer rat thyroid (FRT) cells were cultured in Kaign's modified Ham's F-12 medium supplemented with 10% FBS, 2 mM L-glutamine, 100 units/mL penicillin, 100 μg/mL streptomycin, 18 μg/mL myoinositol, and 45 μg/mL ascorbic acid. To generate FRT cells stably expressing I1234del-, W1282C/L/R and Q1313X-CFTR, cells were transfected with pIRESpuro3-based vectors and clonal cell lines were isolated after inclusion of 0.15 μg/mL puromycin (Invitrogen) in cell culture medium. FRT cell lines expressing wild type, W1282X- and N1303K-CFTR were cultured as reported (Pranke, I. et al., (2019) Emerging therapeutic approaches for cystic fibrosis. From gene editing to personalized medicine. Front. Pharmacol. 10, 121; Phuan, P.-W. et al., (2018) Combination potentiator (‘co-potentiator’) therapy for CF caused by CFTR mutants, including N1303K, that are poorly responsive to single potentiators. J. Cyst. Fibros. 17, 595-606; Cil, O. et al., (2016) CFTR activator increases intestinal fluid secretion and normalizes stool output in a mouse model of constipation. Cell. Mol. Gastroentrol. 2, 317-327). FRT cells lines expressing G85E-, R334W-, R347P-, S492F-, V520F-, R560T-, A561E-, L1077P-, M1101K- and R1162X-CFTR were a generous gift from Dr. Eric Scorcher (Emory University) and cultured as described (Han, S. T. et al., (2018) Residual function of cystic fibrosis mutants predicts response to small molecule CFTR modulators. JCI Insight 3, (14):e121159). Gene edited 16HBE14o-cells expressing N1303K-CFTR were provided by the CFFT Lab, and were cultured as described (Valley, H. C. et al, (2018) Isogenic cell modules of cystic-fibrosis-causing variants in natively expressing pulmonary epithelial cells. J. Cyst. Fibros. 18, 476-483).

### Primary Human Bronchial and Nasal Epithelial Cell Cultures

Human bronchial epithelial cells isolated from a lung transplant from a N1303K homozygous CF subject were provided by Scott H. Randell (Marsico Lung Institute, The University of North Carolina at Chapel Hill, USA). The cells were obtained under protocol #03-1396 approved by the University of North Carolina at Chapel Hill Biomedical Institutional Review Board. Cells were isolated, conditionally reprogrammed, and expanded as described (Haggie, 2017; Fulcher, M. L., and Randell, S. H. (2013) Human nasal and treacho-bronchial respiratory epithelial cell culture. Methods Mol. Biol. 945, 109-121).

### High-Throughput Screening

High-throughput screening used a semi-automated screening platform (Beckman, Fullerton, Calif.) as described (Haggie, 2017). FRT cells expressing W1282X and YFP were plated in 96-well black-walled, clear-bottom tissue culture plates (Corning) at a density of 20,000 cells/well and grown for 24 h at 37° C. to ˜90% confluency. Cells were treated with 3 μM VX-809 for 24 hours. Cells were then washed twice with PBS, and incubated in 100 μl of PBS containing forskolin (10 μM), VX-770 (15 nM) and test compounds (25 μM) for 10 min prior to assay of CFTR activity. All plates contained wells with positive (5 μM VX-770+20 μM ASP-11) and negative (5 μM VX-770) controls. Assays were done using a BMG Labtech FLUOstar OMEGA plate reader (Cary, N.C.) over 12 s with initial fluorescence intensity recorded for 2 s prior to addition of 100 μl of NaI-substituted PBS (137 mM NaCl replaced with NaI). Initial iodide influx was computed from fluorescence intensity by single exponential regression.

### Short-Circuit Current Measurements

Short-circuit current was measured on cells cultured on permeable supports (Corning) as described (Haggie, 2017; Phuan, 2018). For FRT cells, the basolateral membrane was permeabilized with 250 μg/mL amphotericin B, and experiments were done using a HCO₃⁻-buffered system (in mM: 120 NaCl, 5 KCl, 1 MgCl₂, 1 CaCl₂), 5 Hepes, 25 NaHCO₃, 10 glucose, pH 7.4) with a basolateral to apical chloride gradient (60 mM NaCl replaced by sodium gluconate in the apical solution). For human airway epithelial cells, symmetrical HCO₃⁻-buffered solutions (containing 120 mM NaCl) were used. Cells were equilibrated with 95% O₂, 5% CO₂ and maintained at 37° C. Hemichambers were connected to a DVC-1000 voltage clamp (World Precision Instruments Inc., Sarasota, Fla.) via Ag/AgCl electrodes and 3 M KCl agar bridges for recording of the short-circuit current.

### Data Analysis

GraphPad Prism software (GraphPad Inc., San Diego, Calif., USA) was used for statistical analysis. EC₅₀ values were determined by non-linear regression to a single-site inhibition model. Statistical significance was determined using unpaired Student's t test, with P<0.05 considered significant.

### General Experimental for Synthetic Scheme (I)

Synthesis of 2′,3′,4′,9′-tetrahydrospiro[piperidine-4,1′-pyrido[3,4-b]indoles] (8→9): Tryptamine (8, 0.6 mmol) was mixed with glacial acetic acid (3 mL) in a 10 mL vial and stirred with a magnetic stirrer. Corresponding ketone (12, 0.5 mmol) was added and the vial was sealed with a plastic cap. The vial was heated at 100° C. for 16 h in an oil bath. After cooling, the solution was diluted with water (˜20 mL) and neutralized by adding 4 M HCl. The product was extracted with dichloromethane, and the organic solution was washed with water, brine and dried over magnesium sulfate. The solvent was removed in vacuo, and the product was purified by using flash column chromatography (2.5% MeOH/DCM).

Synthesis of N-alkylated piperidin-4-one analogs (11→12): 4-Piperidone hydrochloride (11, 5 mmol) was mixed with 25 mL of dichloromethane in an Erlenmeyer flask. Small amount of methanol (5 drops) was added and benzyl bromide (2.5 mmol) and potassium carbonate (5 mmol) was added. The mixture was stirred at RT for 16 h. Water was added to the reaction mixture and the product was extracted with dichloromethane. The extracted organic solution was washed with water, brine, and dried over magnesium sulfate. The solvent was removed in vacuo, and the product was purified by using flash column chromatography (2.5% MeOH/DCM).

Synthesis of 2′,3′,4′,9′-tetrahydrospiro[piperidine-4,1′-pyrido[3,4-b]indoles] (10→9): Compound 3a (0.5 mmol) was mixed with 5 mL of dichloromethane in a small vial. Small amount of methanol (1 drop) was added for better solubility, and benzyl bromide (0.5 mmol) and potassium carbonate (1.5 mmol) was added. The vial was capped, and the mixture was stirred at RT for 16 h. Water was added to the reaction mixture and the product was extracted with dichloromethane. The extracted organic solution was washed with water, brine, and dried over magnesium sulfate. The solvent was removed in vacuo, and the product was purified by using flash column chromatography (2.5% MeOH/DCM).

Synthesis of 2′,3′,4′,9′-tetrahydrospiro[piperidine-4,1′-pyrido[3,4-b]indoles] (8→10): 5-methoxytryptamine (8, 25 mmol) was mixed with glacial acetic acid (20 mL) in a round bottom flask. 4-piperidone hydrochloride (11, 25 mmol) was added and the solution was heated at 100° C. for 16 h in an oil bath with stirring. The solution was cooled to RT and diluted with water (100 mL) and neutralized with 4 M NaOH. Tan precipitate formed upon standing within an hour. The precipitate was filtered and washed with water and air dried. Yield=54%.

Synthesis of tryptamines (7→8): 1-Dimethylamino-2-nitroethylene (3 mmol) was mixed in TFA (4 mL) in a vial. Substituted indole (7, 3.6 mmol) was dissolved in dichloromethane (3 mL) separately and added. The mixture was stirred at RT for 2 h and the solution was diluted with dichloromethane. The organic solution was washed with water, brine and dried over magnesium sulfate. Solvent was removed in vacuo and purified by using flash column chromatography (50% EtOAc/hexane). LiAlH4 (12 mmol) was mixed with THE (75 mL) and cooled at −78° C. and stirred. The product from the previous step dissolved in small amount of THE was added dropwise and the mixture was stirred overnight with slowly warming to RT. The reaction mixture was cooled in an ice bath and quenched with water and 4 M NaOH solution. Organic solvent was removed in vacuo and the product was extracted with dichloromethane. The extracted solution was washed with water, brine, and dried over magnesium sulfate. The solvent was removed in vacuo, and the product was directly used.

### Example 1

1-(3-Chloro-2,4-difluorobenzyl)-6′-methoxy-2′,3′,4′,9′-tetrahydrospiro[piperidine-4,1′-pyrido[3,4-b]indole] (1j). Yield=124 mg (58%) 1H NMR (400 MHz, CDCl3) δ 8.10 (s, 1H), 7.36-7.24 (m, 1H), 7.17 (s, 1H), 7.01-6.87 (m, 2H), 6.83 (dd, J=8.7, 2.5 Hz, 1H), 3.88 (s, 3H), 3.65 (s, 2H), 3.15 (t, J=5.7 Hz, 2H), 2.80-2.66 (m, 4H), 2.60 (td, J=11.9, 2.6 Hz, 2H), 2.10 (td, J=13.4, 4.6 Hz, 2H), 1.87-1.67 (m, 2H). ¹³C NMR (101 MHz, CDCl3) δ 158.08 (dd, J_{C-F}=250.1, 2.9 Hz), 157.44 (d, J_{C-F}=251.6, 2.9 Hz), 154.01, 140.77, 130.62, 129.34 (dd, J_{C-F}=9.0, 5.7 Hz), 127.74, 121.95 (dd, J_{C-F}=15.0, 4.0 Hz), 111.50, 111.46, 111.44 (dd, J_{C-F}=20.6, 4.4 Hz), 109.77 (t, J_{C-F}=21.3 Hz), 108.58, 100.56, 56.06, 55.41, 50.51, 48.65, 39.10, 36.27, 23.21. HRMS (ESI) m/z for C₂₃H25C₁F2N30 [M+H]. calcd 432.1654, found 432.1646.

### Example 2

6′-Methoxy-1-(3,4,5-trifluorobenzyl)-2′,3′,4′,9′-tetrahydrospiro[piperidine-4,1′-pyrido[3,4-b]indole] (2d). Yield=159 mg (77%).1H NMR (400 MHz, CDCl3) δ 7.76 (s, 1H), 7.23 (d, J=8.6 Hz, 1H), 7.04 (t, J=7.5 Hz, 2H), 6.96 (d, J=2.4 Hz, 1H), 6.84 (dt, J=8.7, 1.8 Hz, 1H), 3.87 (s, 3H), 3.52 (s, 2H), 3.16 (t, J=5.7 Hz, 2H), 2.71 (q, J=5.3 Hz, 4H), 2.56 (t, J=11.8 Hz, 2H), 2.10 (td, J=13.2, 4.3 Hz, 2H), 1.81 (d, J=13.7 Hz, 2H). ¹³C NMR (201 MHz, CDCl3) δ 154.07, 151.14 (ddd, J_{C-F}=249.5, 10.1, 3.7 Hz), 140.68, 139.34-138.02 (m), 135.07, 130.58, 127.74, 112.54 (dd, J_{C-F}=17.1, 3.7 Hz), 111.53, 111.50, 108.64, 100.53, 61.90, 56.05, 50.55, 48.79, 39.08, 36.34, 23.19. HRMS (ESI) m/z for C₂₃H24F3N30 [M+H]. calcd 416.1950, found 416.1939.

### Example 3

6′-Methoxy-1-((perfluorophenyl)methyl)-2′,3′,4′,9′-tetrahydrospiro[piperidine-4,1′-pyrido[3,4-b]indole] (2e). Yield=202 mg (90%).1H NMR (400 MHz, CDCl3) δ 7.73 (s, 1H), 7.19 (d, J=8.7 Hz, 1H), 6.95 (t, J=1.8 Hz, 1H), 6.83 (dt, J=8.7, 1.9 Hz, 1H), 3.87 (d, J=1.4 Hz, 3H), 3.79 (d, J=2.4 Hz, 2H), 3.13 (t, J=5.7 Hz, 2H), 2.76 (d, J=11.1 Hz, 2H), 2.72-2.61 (m, 4H), 2.06 (td, J=13.1, 4.5 Hz, 2H), 1.80 (d, J=13.6 Hz, 2H). ¹³C NMR (201 MHz, CDCl3) δ 154.03, 145.63 (d, J_{C-F}=248.0 Hz), 140.70, 140.59 (d, J_{C-F}=254.2 Hz), 137.39 (d, J_{C-F}=251.6 Hz), 130.54, 127.73, 111.47, 111.46, 110.75 (t, J_{C-F}=18.7 Hz), 108.66, 100.50, 56.01, 50.26, 48.92, 48.07, 39.04, 36.34, 23.20. HRMS (ESI) m/z for C23H23F5N30 [M+H]. calcd 452.1761, found 452.1753.

### Example 4

6′-Methoxy-1-(2,3,4-trifluorobenzyl)-2′,3′,4′,9′-tetrahydrospiro[piperidine-4,1′-pyrido[3,4-b]indole] (2g). Yield=200 mg (96%) 1H NMR (400 MHz, CDCl3) δ 8.24 (s, 1H), 7.18 (d, J=8.8 Hz, 1H), 7.15-7.03 (m, 1H), 6.99-6.86 (m, 2H), 6.85-6.67 (m, 1H), 3.87 (d, J=2.9 Hz, 3H), 3.68-3.59 (m, 2H), 3.13 (t, J=5.7 Hz, 2H), 2.77-2.65 (m, 4H), 2.65-2.50 (m, 2H), 2.16-1.98 (m, 2H), 1.85-1.71 (m, 2H). ¹³C NMR (101 MHz, CDCl3) δ 153.99, 150.40 (ddd, J_{C-F}=250.2, 9.9, 2.6 Hz), 150.20 (d, J_{C-F}=250.4, 9.5, 3.0 Hz), 140.80, 139.88 (dt, J_{C-F}=251.8, 15.6 Hz), 130.65, 127.72, 124.92 (ddd, J_{C-F}=8.5, 6.3, 3.2 Hz), 122.42 (dd, J_{C-F}=12.1, 2.8 Hz), 111.71 (dd, J_{C-F}=17.1, 3.9 Hz), 111.51, 111.42, 108.52, 100.53, 56.05, 55.14, 50.49, 48.58, 39.08, 36.21, 23.20. HRMS (ESI) m/z for C₂₃H24F3N30 [M+H]. calcd 416.1950, found 416.1941.

### Example 5

6′-Methoxy-1-(2,4,5-trifluorobenzyl)-2′,3′,4′,9′-tetrahydrospiro[piperidine-4,1′-pyrido[3,4-b]indole] (2i). Yield=193 mg (93%) 1H NMR (400 MHz, CDCl3) δ 8.06 (s, 1H), 7.30 (ddd, J=10.7, 8.9, 6.6 Hz, 1H), 7.19 (d, J=8.6 Hz, 1H), 6.98 (d, J=2.4 Hz, 1H), 6.95-6.86 (m, 1H), 6.84 (dd, J=8.7, 2.5 Hz, 1H), 3.89 (s, 3H), 3.59 (d, J=1.3 Hz, 2H), 3.16 (t, J=5.7 Hz, 2H), 2.81-2.66 (m, 4H), 2.60 (dd, J=23.9, 2.7 Hz, 2H), 2.10 (td, J=13.7, 4.6 Hz, 2H), 1.80 (dd, J=14.1, 2.7 Hz, 2H). ¹³C NMR (101 MHz, CDCl3) δ 156.20 (ddd, J_{C-F}=247.5, 10.1, 2.2 Hz), 154.03, 149.1 (ddd, J_{C-F}=251.7, 14.9, 14.0), 146.8 (ddd, J_{C-F}=245.3, 12.5, 4.0), 140.79, 130.64, 127.76, 121.72 (dt, J_{C-F}=16.9, 4.6 Hz), 118.72 (dd, J_{C-F}=18.9, 5.7 Hz), 111.52, 111.47, 108.60, 105.31 (dd, J_{C-F}=28.6, 20.5 Hz), 100.57, 56.06, 54.76, 50.52, 48.70, 39.09, 36.34, 23.21. HRMS (ESI) m/z for C₂₃H24F3N30 [M+H]. calcd 416.1950, found 416.1940.

### Example 6

6′-Chloro-1-(2,4-difluorobenzyl)-2′,3′,4′,9′-tetrahydrospiro[piperidine-4,1′-pyrido[3,4-b]indole](5c). Yield=134 mg (67%).1H NMR (400 MHz, CDCl3) δ 8.86-8.49 (m, 1H), 7.43 (d, J=9.3 Hz, 2H), 7.21 (d, J=8.7 Hz, 1H), 7.08 (d, J=8.5 Hz, 1H), 6.82 (q, J=9.0 Hz, 2H), 3.69 (s, 2H), 3.11 (d, J=5.8 Hz, 2H), 2.81 (d, J=11.3 Hz, 2H), 2.67 (q, J=8.5, 8.0 Hz, 4H), 2.18 (q, J=8.0, 5.3 Hz, 2H), 1.76 (d, J=13.6 Hz, 2H). ¹³C NMR (101 MHz, CDCl3) δ 163.25 (dd, J_{C-F}=100.2, 12.4 Hz), 160.77 (dd, J_{C-F}=100.6, 12.3 Hz), 141.11, 133.91, 132.90 (dd, J_{C-F}=9.8, 5.8 Hz), 128.44, 124.90, 121.74, 119.48 (d, J_{C-F}=15.4 Hz), 117.69, 111.85, 111.29 (d, J_{C-F}=20.7 Hz), 108.51, 103.82 (t, J_{C-F}=25.7 Hz), 54.96, 50.36, 48.43, 39.00, 35.72, 22.99. HRMS (ESI) m/z for C₂₂H23C₁F2N3 [M+H]. calcd 402.1549, found 402.1539.

### Example 7

1-(2,4-Difluorobenzyl)-7′-methoxy-2′,3′,4′,9′-tetrahydrospiro[piperidine-4,1′-pyrido[3,4-b]indole] (5d). Yield=90 mg (45%).1H NMR (400 MHz, CDCl3) δ 8.24 (s, 1H), 7.48-7.32 (m, 2H), 6.94-6.68 (m, 4H), 3.82 (s, 3H), 3.67 (s, 2H), 3.13 (t, J=5.7 Hz, 2H), 2.83-2.76 (m, 2H), 2.72-2.54 (m, 4H), 2.17 (td, J=13.5, 13.1, 4.4 Hz, 2H), 1.77 (d, J=13.7 Hz, 2H). ¹³C NMR (101 MHz, CDCl3) δ 163.17 (dd, J_{C-F}=91.6, 11.9 Hz), 160.70 (dd, J_{C-F}=92.1, 12.1 Hz), 156.28, 138.34, 136.25, 132.75 (dd, J_{C-F}=9.5, 5.9 Hz), 121.77, 119.96 (dd, J_{C-F}=14.8, 3.8 Hz), 118.65, 111.20 (dd, J_{C-F}=20.8, 3.8 Hz), 108.93, 108.49, 103.74 (t, J_{C-F}=25.7 Hz), 94.99, 55.78, 54.99, 50.38, 48.59, 39.15, 35.97, 23.14. HRMS (ESI) m/z for C₂₃H26F2N30 [M+H]. calcd 398.2044, found 398.2033.

### Example 8

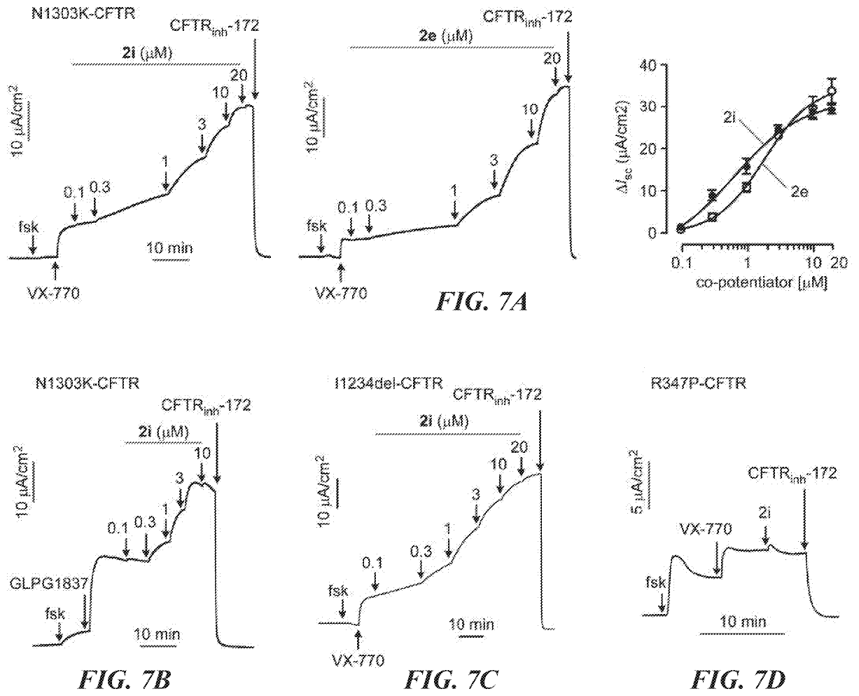

Additional biological studies were performed for most potent Compounds 2i and 2e. Co-potentiator efficacy was initially determined by short-circuit current measurements in FRT cells expressing N1303K-CFTR in the presence of a transepithelial chloride gradient and with permeabilization of the basolateral cell membrane such that measured current directly reports CFTR channel activity. Representative data in FIG. 7A show small increases in CFTR activity following addition of the cAMP agonist forskolin and the potentiator VX-770, followed by concentration-dependent increases in current following addition of the co-potentiators 2i (left) and 2e (middle), with EC₅₀ values 0.6±0.2 and 2.1±0.3 μM, respectively (right). We previously defined potentiators as either class I compounds, including VX-770 and GLPG1837, or class II compounds such as the arylsulphonamide-pyrrolopyridine and spiro[piperidine-4,1-pyrido[3,4-b]indole] copotentiators that probably bind at distinct sites on CFTR. N1303K-CFTR expressing FRT cells treated with forskolin and GLPG1837 had similar 2i co-potentiator EC₅₀ of 0.8±0.2 μM (FIG. 7B).

Compound 2i was also tested on a second minimal function CFTR mutation, I1234del-CFTR, which is generated by the c.3700A>G mutation that results in deletion of 6 amino acids from the CFTR polypeptide (p.Ile1234_Arg1239del-CFTR, hereafter termed I1234del-CFTR) due to introduction of a cryptic splice site in the CFTR transcript. As seen in FIG. 7C, 2i further activates I1234del-CFTR following forskolin and VX-770 to increase channel activity with EC₅₀ of 0.7±0.2 μM, similar to that found with N1303K-CFTR. The R347P-CFTR mutant, which is mildly responsive to VX-770 but not responsive to arylsulphonamide-pyrrolopyridine co-potentiators, was not activated by 2i, indicating that alternative mechanisms (such as increased cAMP signaling) are not responsible for activation of the N1303K- and I1234del-CFTR mutants (FIG. 7D).

### Example 9

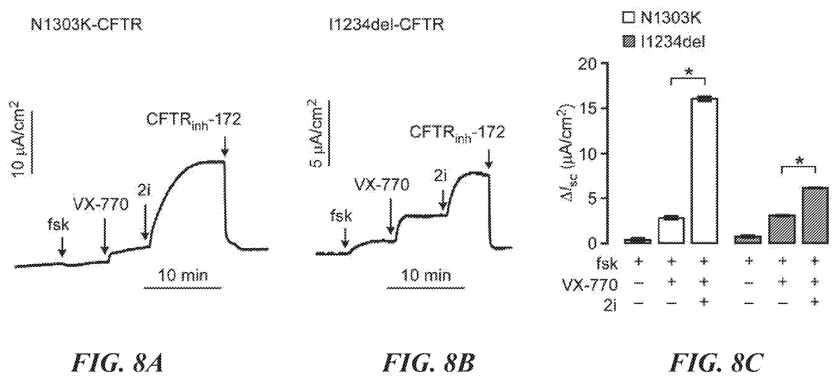

The efficacy of Compound 2i was also tested in 16HBE14o-human bronchial epithelial cell models in which the endogenous CFTR gene was edited to contain the N1303K mutation (16HBEge-N1303K) or the I1234del mutation (16HBEge-I1234del). As shown for N1303K- (FIG. 8A) and I1234del-CFTR (FIG. 8B) expressing 16HBE14o-cells, addition of forskolin and then VX-770 produced limited channel activity. However, subsequent addition of Compound 2i produced CFTRᵢₙₕ-172-inhibitable responses of 16.1±0.4 μA/cm2 in N1303K- (FIG. 8A) and 6.1±0.1 μA/cm2 in I1234del-CFTR (FIG. 8B) expressing cells, approximately 6-fold and 2-fold greater that that produced by VX-770 alone.

### REFERENCES

1. Elborn, J. S. (2016) Cystic Fibrosis. Lancet 388, 2519-2531
2. Burgener, E. B., and Moss, R. B. (2018) Cystic fibrosis transmembrane conductance regulator modulators: precision medicine in cystic fibrosis. Curr. Opin. Pediatr. 30, 372-377
3. Clancy, J. P. (2018) Rapid therapeutic advances in CFTR modulator science. Pediatr. Pulmonol. 53, S4-S11
4. Kym, P. R., Wang, X., Pizzonero, M., and Van der Plas, S. E. (2018) Recent progress in the discovery and development of small-molecule modulators of CFTR. Prog. Med. Chem. 57, 235-276
5. Keating, D., Marigowda, G., Burr, L., Daines, C., Mall, M. A., McKone, E. F., Ramsey, B. W., Rowe, S. M., Sass, L. A., Tullis, E., McKee, C. M., Moskowitz, S. M., Robertson, S., Savage, J., Simard, C., Van Goor, F., Waltz, D., Xuan, F., Young, T., Taylor-Cousar, J. L.; VX16-445-001 Study Group (2018) VX-445-Tezacaftor-Ivacaftor in patients with cystic fibrosis and one or two Phe508del alleles. N. Engl. J. Med. 379, 1612-1620
6. Davies, J. C., Moskowitz, S. M., Brown, C., Horsley, A., Mall, M. A., McKone, E. F., Plant, B. J., Prais, D., Ramsey, B. W., Taylor-Cousar, J. L., Tullis, E., Uluer, A., McKee, C. M., Robertson, S., Shilling, R. A., Simard, C., Van Goor, F., Waltz, D., Xuan, F., Young, T., Rowe, S. M.; VX16-659-101 Study Group (2018) VX-659-Tezacaftor-Ivacaftor in patients with cystic fibrosis and one or two Phe508del alleles. N. Engl. J. Med. 379, 1599-1611
7. Cabrini, G. (2019) Innovative therapies for cystic fibrosis: the road from treatment to cure. Mol. Diagn. Ther. 23, 263-279
8. Pranke, I., Golec, A., Hinzpeter, A., Edelman, A., and Semet-Gaudelus, I. (2019) Emerging therapeutic approaches for cystic fibrosis. From gene editing to personalized medicine. Front. Pharmacol. 10, 121
9. Haggie, P. M., Phuan, P.-W., Tan, J. A., Xu, H., Avramescu, R. G., Perdomo, D., Zlock, L., Nielson, D. W., Finkbeiner, W. E., Lukacs, G. L., and Verkman, A. S. (2017) Correctors and potentiators rescue function of the truncated W1282X-cystic fibrosis transmembrane regulator (CFTR) translation product. J. Biol. Chem. 292, 771-785
10. Phuan, P.-W., Son, J.-H., Tan, J. A., Li, C., Musante, I., Zlock, L., Nielson, D. W., Finkbeiner, W. E., Kurth, M. J., Galietta, L. J., Haggie, P. M., and Verkman, A. S. (2018) Combination potentiator (‘co-potentiator’) therapy for CF caused by CFTR mutants, including N1303K, that are poorly responsive to single potentiators. J. Cyst. Fibros. 17, 595-606
11. Molinski, S. V., Gonska, T., Huan, L. J., Baskin, B., Janahi, I. A., Ray, P. N., and Bear, C. E. (2014) Genetic, cell biological, and clinical interrogation of the CFTR mutation c.3700 A>G (p.Ile1234V) informs of strategies for future medical intervention. Genetics Med. 16, 625-632
12. Cui, L., Aleksandrov, L. A., Chang, X.-B., Hou, Y.-X., He, L., Hegedus, T., Gentzsch, M., Aleksandrov, A. A., Balch, W., and Riordan, J. R. (2007) Domain interdependence in the biosynthetic assembly of CFTR. J. Mol. Biol. 365, 981-994
13. Du, K., and Lukacs, G. L. (2009) Cooperative assembly and misfolding of CFTR domains in vivo. Mol. Biol. Cell 20, 1903-1915
14. Zomer-van Ommen, D. D., Vijfitigschild, L. A. W., Kruisselbrink, E., Vonk, A. M., Dekkers, J. F., Janssens, H. M., de Winter-de Groot, K. M., van der Ent, C. K., and Beekman, J. M. (2016) Limited premature termination codon suppression by read-through agents in cystic fibrosis intestinal organoids. J. Cyst. Fibros. 15, 158-162
15. Mutyam, V., Du, M., Xue, X., Keeling, K. M., White, E. L., Bostwick, J. R., Rasmussen, L., Liu, B., Mazur, M., Hong, J. S., Falk Libby, E., Liang, F., Shang, H., Mense, M., Suto, M. J., Bedwell, D. M., and Rowe, S. M. (2016) Discovery of clinically approved agents that promote suppression of cystic fibrosis transmembrane conductance regulator nonsense mutations. Am. J. Respir. Crit. Care Med. 194, 1092-1103
16. Roy, B., Friesen, W. J., Tomizawa, Y., Leszyk, J. D., Zhuo, J., Johnson, B., Dakka, J., Trotta, C. R., Xue, X., Mutyam, V., Keeling, K. M., Mobley, J. A., Rowe, S. M., Bedwell, D. M., Welch, E. M., and Jacobson, A. (2016) Ataluren stimulates ribosomal selection of near-cognate tRNAs to promote nonsense suppression. Proc. Natl. Acad. Sci. USA 113, 12508-12513
17. Kerem, E., Konstan, M. W., De Boeck, K., Accruso, F. J., Sermet-Gaudelus, I., Wilschanski, M., Elborn, J. S., Melotti, P., Bronsveld, I., Fajac, I., Malfroot, A., Rosenbluth, D. B., Walker, P. A., McColley, S. A., C., K., Quattrucci, S., Rietschel, E., Zeitlin, P. L., Barth, J., Elfring, G. L., Welch, E. M., Branstrom, A., Spiegel, R. J., Peltz, S. W., Ajayi, T., Rowe, S. M., and Group, C. F. A. S. (2014) Ataluren for the treatment of nonsense-mutation cystic fibrosis: a randomized, double-blind, placebo-controlled phase 3 trial. Lancet Respir. Med. 2, 539-547
18. Xue, X., Mutyam, V., Thakerar, A., Mobley, J., Bridges, R. J., Rowe, S. M., Keeling, K. M., and Bedwell, D. M. (2017) Identification of the amino acids inserted during suppression of CFTR nonsense mutations and determination of their functional consequences. Hum. Mol. Genet. 26, 3116-3129
19. Yeh, H-I., Sohma, Y., Conrath, K., and Hwang, T.-C. (2017) A common mechanism for CFTR potentiators. J. Gen. Physiol. 149, 1105-1118
20. Valley, H. C., Bukis, K. M., Bell, A., Cheng, Y., Wong, E., Jordan, N. J., Allaire, N. E., Sivachenko, A., Liang, F., Bihler, H., Thomas, P. J., Mahlou, J., and Mense, M. (2018) Isogenic cell models of cystic-fibrosis-causing variants in natively expressing pulmonary epithelial cells. J. Cyst. Fibros. 18, 476-483
21. Strug, L. J., Stephenson, A. L., Panjwani, N., and Harris, A. (2018) Recent advances in developing therapeutics for cystic fibrosis. Hum. Mol. Genet. 27
22. Hwang, T.-C., Yeh, J. T., Zhang, J., Yu, Y. C., Yeh, H. I., and Destefano, S. (2018) Structural mechanism of CFTR function and dysfunction. J. Gen. Physiol. 150, 539-570
23. Yeh, H. I., Qui, L., Sohma, Y., Conrath, K., Zou, X., and Hwang, T. C. (2019) Identifying the molecular target sites for CFTR potentiators GLPG1837 and VX-770. J. Gen. Physiol. In press
24. Liu, F., Zhang, Z., Levit, A., Levring, J., Touhara, K. K., Shoichet, B. K., and Chen, J. (2019) Structural identification of a hotspot on CFTR for potentiation. Science 364, 1184-1188
25. Han, S. T., Rab, A., Pellicore, M. J., Davis, E. F., McCague, A. F., Evans, T. A., Joynt, A. T., Lu, Z., Cai, Z., Raraigh, K. S., Hong, J. S., Sheppard, D. N., Sorscher, E. J., and Cutting, G. R. (2018) Residual function of cystic fibrosis mutants predicts response to small molecule CFTR modulators. JCI Insight 3, (14):e121159
26. Veit, G., Avramescu, R. G., Chiang, A. N., Houck, S. A., Cai, Z., Peters, K. W., Hong, J. S., Pollard, H. B., Guggino, W. B., Balch, W. E., Skach, W. R., Cutting, G. R., Frizzell, R. A., Sheppard, D. N., Cyr, D. M., Sorscher, E. J., Brodsky, J. L., and Lukacs, G. L. (2016) From CFTR biology towards combinatorial pharmacotherapy: expanded classification of cystic fibrosis mutations. Mol. Biol. Cell 27, 424-433
27. Banjar, H., and Angyalosi, G. (2015) The road for survival improvement of cystic fibrosis patients in Arab countries. Int. J. Pediatr. Adolesc. Med. 2, 47-58
28. Al-Sadeq, D., Abunada, T., Dalloul, R., Fahad, S., Taleb, S., Aljassim, K., Al Hamed, F. A., and Zayed, H. (2019) Spectrum of mutations of cystic fibrosis in the 22 Arab countries: A systematic review. Respirology 24, 127-136
29. Kervestin, S., and Jacobson, A. (2012) NMD: a multifacted response to premature translation termination. Nat. Rev. Mol. Cell Biol. 13, 700-712
30. Pop, M. W.-L., and Maquat, L. E. (2013) Organizing principles of mammalian nonsense-mediated mRNA decay. Annu. Rev. Genet. 47, 139-165
31. Linde, L., Boelz, S., Nissim-Rafina, M., Oren, Y. S., Wilschanski, M., Yaacov, Y., Virgilis, D., Neu-Yilik, G., Kulozik, A. E., Kerem, E., and Kerem, B. (2007) Nonsense-mediated mRNA decay affects nonsense transcript levels and governs response of cystic fibrosis patients to gentamicin. J. Clin. Invest. 117, 683-692
32. Clarke, L. A., Awatade, N. T., Felicio, V. M., Silva, I. A., Calucho, M., Pereira, L., Azevedo, P., Cavaco, J., Barreto, C., Bertuzzo, C., Gartner, S., Beekman, J. M., and Amaral, M. D. (2019) The effect of premature termination codon mutations on CFTR mRNA abundance in human nasal epithelium and intestinal organoids: a basis for read-through therapies in cystic fibrosis. Hum. Mut. 40, 326-334
33. Aksit, M. A., Bowling, A. D., Evans, T. A., Joynt, A. T., Osorio, D., Patel, S., West, N., Merio, C., Sosnoy, P. R., Cutting, G. R., and Sharma, N. (2019) Decreased mRNA and protein stability of W1282X limits response to modulator therapy. J. Cyst. Fibros. In press
34. Lapa, G. B., Bekker, O. B., Mirchink, E. P., Danilenko, V. N., and Preobrazhenskaya, M. N. (2013) Regioselective acylation of congeners of 3-amino-1H-pyrazolo[3,4-b]quinolines, their activity on bacterial serine/threonin protein kinases and in vitro antibacterial (including antimycobacterial) activity. J. Enzyme. Inhib. Med. Chem. 28, 1088-1093
35. Gilleron, J., Paramasivam, P., Zeigerer, A., Querbes, W., Marsico, G., Andree, C., Seifert, S., Amaya, P., Stoter, M., LKoteliansky, V., Waldmann, H., Fitzgerald, K., Kalaidzidis, Y., Akinc, A., Maier, M. A., Manoharan, M., Bickle, M., and Zerial, M. (2015) Identification of siRNA delivery enhancers by a chemical library screen. Nuc. Acids Res. 18, 7984-8001
36. Mokrosz, M. J., Duszynska, B., Bojarski, A. J., and Mokrosz, J. L. (1995) Structure-activity relationship studies of CNS agents—XVII. Spiro[piperidine-4′,1-(1,2,3,4-tetrahydro-beta-carboline)] as a probe defining the extended topographic model of 5-HT1A receptors. Bioorg. Med. Chem. 3, 533-538
37. Bartyzel, P., Misztal, S., Tatarczynska, E., and Chojnacka-Wojcik, E. (1989) N-aminoalkyl derivatives of 1,2,3,4-tetrahydro-beta-carboline-1-spiro-4′-N′-benzylpiperidine—a putative way to novel anxiolytic agents. Pol. J. Pharmacol. Pharm. 41, 495-504
38. Pedemonte, N., Sonawane, S. D., Taddei, A., Hu, J., Zegarra-Moran, O., Suen, Y. F., Robins, L. I., Dicus, C. W., Willenbring, D., Nantz, M. H., Kurth, M. J., Galietta, L. J., and Verkman, A. S. (2005) Phenylglycine and sulfonamide correctors of defective delta F508 and G551D cystic fibrosis transmembrane conductance regulator chloride-channel gating. Mol. Pharmacol. 67, 1979-1807
39. Yang, H., Shelat, A. A., Guy, R. K., Gopinath, V. S., Ma, T., Du, K., Lukacs, G. L., Taddei, A., Folli, C., Pedemonte, N., Galietta, L. J. V., and Verkman, A. S. (2003) Nanomolar-affinity small-molecular potentiators of DF508-CFTR chloride channel gating. J. Biol. Chem. 278, 35079-35085
40. Cil, O., Phuan, P.-W., Lee, S., Tan, J. A., Haggie, P. M., Levin, M. H., Sun, L., Thiagarajah, J. R., Ma, T., and Verkman, A. S. (2016) CFTR activator increases intestinal fluid secretion and normalizes stool output in a mouse model of constipation. Cell. Mol. Gastroentrol. 2, 317-327
41. Fulcher, M. L., and Randell, S. H. (2013) Human nasal and treacho-bronchial respiratory epithelial cell culture. Methods Mol. Biol. 945, 109-121

The various embodiments described above can be combined to provide further embodiments. All U.S. patents, U.S. patent application publications, U.S. patent applications, foreign patents, foreign patent applications, and non-patent publications referred to in this specification and/or listed in the Application Data Sheet are incorporated herein by reference, in their entirety. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

This application claims the benefit of priority to U.S. Provisional Application No. 62/893,107 filed Aug. 28, 2019, the entirety of which is incorporated by reference herein.

1. A combination treatment method for treating cystic fibrosis (CF) in a CF subject having one or more CFTR missense, nonsense and deletion mutations, wherein the combination treatment method comprises administering a Class I potentiator combined with at least one Class II potentiator having one of the structures (A), (B), (C), or (D): 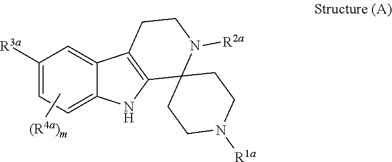
wherein:
m is 0, 1, 2 or 3;
R^{1a} is optionally substituted arylalkyl, optionally substituted heteroaryl or optionally substituted heteroarylalkyl;
R^{2a} is H or C₁-C₆ alkyl;
R^{3a} is H, halo or C₁-C₆ alkoxy; and
R^{4a} is H, C₁-C₆ alkoxy or C₁-C₆ alkyl.
or 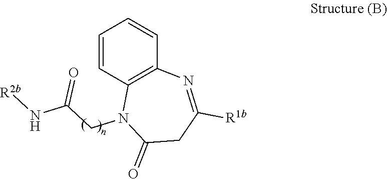
wherein:
n is 1 or 2;
R^{1b} is a 5- or 6-membered heteroaryl; and
R^{2b} is an optionally substituted arylalkyl;
or 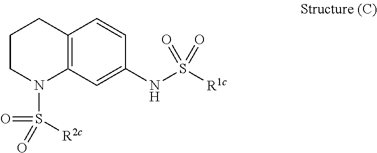
wherein:
R^{1c} is a 5- or 6-membered heteroaryl; and
R^{2c} is C₁-C₆ alkyl.
or 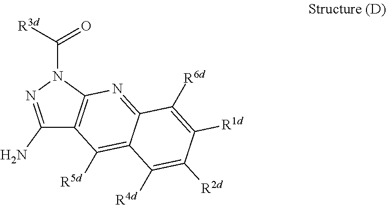
wherein:
R^{1d} is H, C₁-C₆ alkyl, or C₁-C₆ alkoxy;
R^{2d} is H or C₁-C₆ alkoxy;
R^{3d} is substituted aryl, substituted heteroaryl; and
R^{4d}, R^{5d}, and R^{6d} are independently H, C₁-C₆ alkyl, or C₁-C₆ alkoxy.

2. The combination treatment method of claim 1, wherein the CF subject has one or more NBD2 mutations.

3. The combination treatment method of claim 2, wherein the NBD2 mutations are N1303K, W1282X, G551D, I1234del-CFTR, Q1313X, or c.3700 A>G.

4. The combination treatment method of claim 3, wherein the NBD2 mutations are N1303K, W1282X, or G551D.

5. The combination treatment method of claim 1, wherein the Class I potentiator is VX-770, P2, P3, P5, or GLPG1837.

6. The combination treatment method of claim 5, wherein the Class I potentiator is VX-770.

7. The combination treatment method of claim 1, wherein R^{1a} is benzyl or benzyl substituted with one or more substituents selected from the group consisting of halo, and C₁-C₆ alkoxy.

8. The combination treatment method of claim 7, wherein R^{1a} is benzyl, 3-methoxy-benzyl, 2,4-difluoro-benzyl, 3,4-difluoro-benzyl, 3-chloro-2,4-difluoro-benzyl, 3,4,5-trifluoro-benzyl, perfluoro-benzyl, 2,3,4-trifluoro-benzyl, or 2,4,5-trifluoro-benzyl.

9.-10. (canceled)

11. The combination treatment method of claim 1, wherein m is 1, R^{2a} is H, and R^{4a} is methoxy.

12. The combination treatment method of claim 1, wherein R^{1d} is H, methoxy or methyl.

13. The combination treatment method of claim 11, wherein R^{2d} is H or methoxy.

14. The combination treatment method of claim 12, wherein R^{3d} is substituted phenyl having one or more substituents selected from the group consisting of halo, C₁-C₆ alkoxy, C₁-C₆ alkyl, nitro, and heteroaryl.

15.-16. (canceled)

17. The combination treatment method of claim 1, wherein the Class II potentiator is selected from the group consisting of:

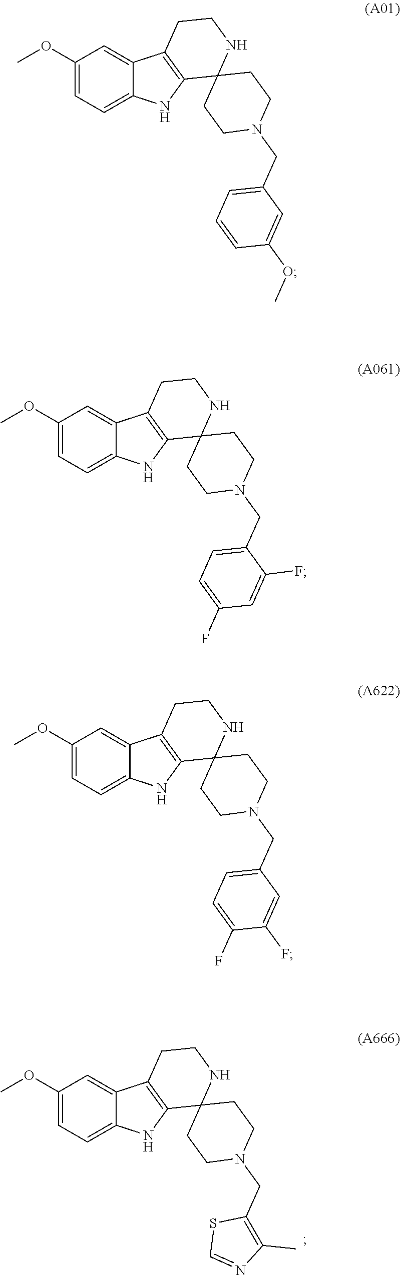

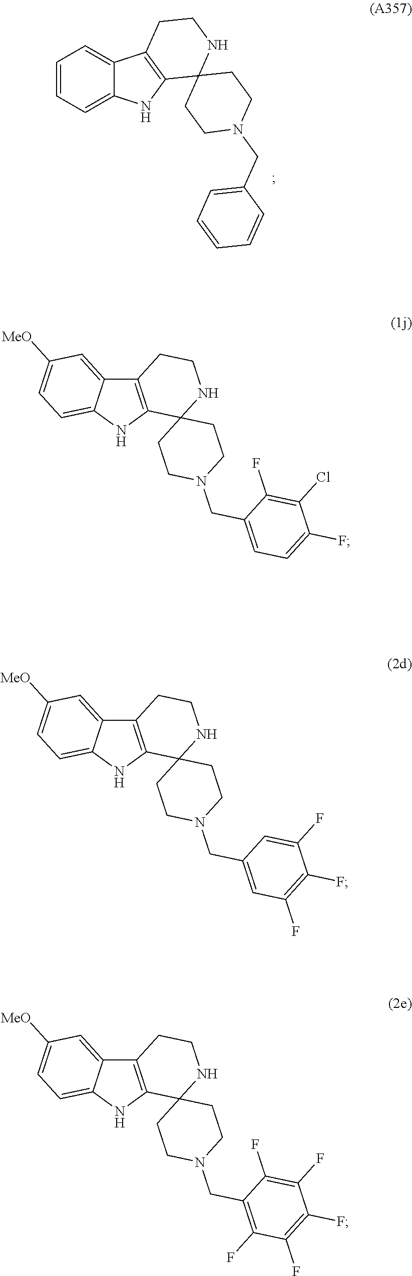 [SMILES: c1ccc(CN2CCC3(CC2)NCCc2c3[nH]c3ccccc23)cc1 ; COc1ccc2[nH]c3c(c2c1)CCNC31CCN(Cc2ccc(F)c(Cl)c2F)CC1 ; COc1ccc2[nH]c3c(c2c1)CCNC31CCN(Cc2cc(F)c(F)c(F)c2)CC1 ; COc1ccc2[nH]c3c(c2c1)CCNC31CCN(Cc2c(F)c(F)c(F)c(F)c2F)CC1]

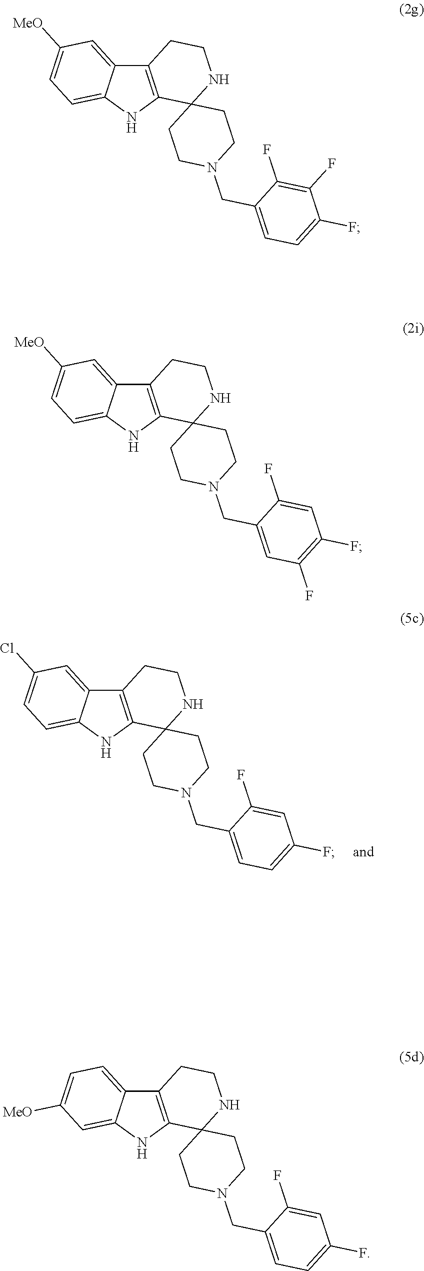 [SMILES: COc1ccc2[nH]c3c(c2c1)CCNC31CCN(Cc2ccc(F)c(F)c2F)CC1 ; COc1ccc2[nH]c3c(c2c1)CCNC31CCN(Cc2cc(F)c(F)cc2F)CC1 ; Fc1ccc(CN2CCC3(CC2)NCCc2c3[nH]c3ccc(Cl)cc23)c(F)c1 ; COc1ccc2c3c([nH]c2c1)C1(CCN(Cc2ccc(F)cc2F)CC1)NCC3]

18. The combination treatment method of claim 1, wherein Class I potentiator and the Class II potentiator are used simultaneously.

19. The combination treatment method of claim 1 further comprising an additional CFTR modulator.

20. The combination treatment method of claim 19, wherein the additional CFTR modulator is a corrector selected from the group consisting of VX-809, VX-661, VX-983, VX-152, VX-440, VX-445, VX-659, GLPG2222, GLPG3221, GLPG2737, GLPG2851, GLPG2665 and a combination thereof, an amplifier, a read-through agent, or a combination thereof.

21. (canceled)

22. The combination treatment method of claim 20, wherein the amplifier is PTI-428.

23. The combination treatment method of claim 20, wherein the read-through agent is ataluren.

24. The combination treatment method of claim 1, wherein the combination further comprises an additional therapeutic agent.

25. The combination treatment method of claim 24, wherein the additional therapeutic agent is NMD inhibitor.

26. (canceled)
